(19) United States
(12) Patent Application Publication
(10) Pub. No.: US 2025/0146021 A1
(43) Pub. Date: May 8, 2025

(54) FOLDING MRNA INTO A NANOSCALE DELIVERY VEHICLE

(71) Applicant: ARIZONA BOARD OF REGENTS ON BEHALF OF ARIZONA STATE UNIVERSITY, Scottsdale, AZ (US)

(72) Inventors: Nicholas Stephanopoulos, Scottsdale, AZ (US); Karen Sue Anderson, Scottsdale, AZ (US); Petr Sulc, Scottsdale, AZ (US); Skyler Jennifer Wray Henry, Tempe, AZ (US); Liangxiao Chen, Tempe, AZ (US); Erik Poppleton, Phoenix, AZ (US); Hao Yan, Chandler, AZ (US)

(21) Appl. No.: 18/726,078

(22) PCT Filed: Jan. 9, 2023
(86) PCT No.: PCT/US2023/060296

§ 371 Date: Jul. 1, 2024

Related U.S. Application Data
(60) Provisional application No. 63/298,096, filed on Jan. 10, 2022.

(51) Int. Cl. C12N 15/87; C12N 15/11; C12N 15/67

(52) U.S. Cl. CPC C12N 15/87; C12N 15/11; C12N 15/67

(57) ABSTRACT

Described herein is a DNA nanotechnology-based platform for the delivery of nucleic acids into cells. The delivery platform relies on compacting cargo mRNA and/or ssDNA into single folded nanostructures that act as the primary structural vehicle material for nanoparticle delivery to cells. The described compositions and methods provide an alternative to packaging mRNA or ssDNA into lipid nanoparticle vehicles, which have complex formulation properties. Also described herein are strategies for protecting mRNA and/or ssDNA cargo and helping these nucleic acid molecules escape the endosome into the cytoplasm using a cationic peptide-PEG coating.

### CROSS-REFERENCE TO RELATED APPLICATIONS

This application claims priority to U.S. Provisional Patent Application No. 63/298,096, filed on Jan. 10, 2022, which is incorporated by reference herein in its entirety.

### FEDERALLY SPONSORED RESEARCH

This invention was made with government support under grant number GM132931 awarded by the National Institutes of Health. The government has certain rights in the invention.

### REFERENCE TO SEQUENCE LISTING

This application was filed with a Sequence Listing XML in ST.26 XML format accordance with 37 C.F.R. § 1.831. The Sequence Listing XML file submitted in the USPTO Patent Center, “208192-9115-WO01_sequence_listing_xml_14 Dec. 2022.xml,” was created on Dec. 14, 2022, contains 81 sequences, has a file size of 126 Kbytes, and is incorporated by reference in its entirety into the specification.

### TECHNICAL FIELD

Described herein is a DNA nanotechnology-based platform for the delivery of nucleic acids into cells. The delivery platform relies on compacting cargo mRNA and/or ssDNA into single folded nanostructures that act as the primary structural vehicle material for nanoparticle delivery to cells. The described compositions and methods provide an alternative to packaging mRNA or ssDNA into lipid nanoparticle vehicles, which have complex formulation properties. Also described herein are strategies for protecting mRNA and/or ssDNA cargo and helping these nucleic acid molecules escape the endosome into the cytoplasm using a cationic peptide-PEG coating.

### BACKGROUND

There is an increased interest in mRNA delivery for therapeutics and vaccination, as seen with the Pfizer and Moderna COVID-19 vaccines. There is also much interest in mRNA delivery for biological research, as CRISPR/Cas9 gene editing technologies highlight the need for efficient gene delivery systems. These current technologies rely on the use of additional lipid nanoparticles, which have complex formulation properties, are expensive to manufacture, and have lower scalability potential.

Thus, what is needed is a new DNA-nanotechnology-based platform for delivering nucleic acid (e.g., mRNA and/or ssDNA) nanostructures to cells that can provide an easier formulation, lower costs, greater scalability potential, a specific cell targeting mechanism, and an endosomal escape mechanism to enhance the efficiency and efficacy of nucleic acid delivery.

### SUMMARY

One embodiment described herein is a nanoparticle composition comprising a folded monodispersed nucleic acid nanostructure. In one aspect, the nucleic acid nanostructure comprises a messenger RNA (mRNA), a single-stranded DNA (ssDNA), or a combination thereof. In another aspect, the nucleic acid nanostructure comprises one or more nucleic acid sequences having at least 90-95% identity to SEQ ID NO: 1, 3, 5, 7, 9, 11, 13, or 15-76. In another aspect, the nucleic acid nanostructure comprises one or more nucleic acid sequences of SEQ ID NO: 1, 3, 5, 7, 9, 11, 13, or 15-76. In another aspect, the composition further comprises one or more DNA or RNA staple strands. In another aspect, the staple strands are short DNA staple strands.

In another aspect, the nucleic acid nanostructure comprises a single-stranded mRNA that serves as a scaffold strand, and one or more short DNA or RNA staple strands that promote folding and stabilization of the mRNA into a compact monodispersed nanostructure. In another aspect, the folded monodispersed nucleic acid nanostructure comprises a 6-helix bundle (6HB), a 12-helix bundle, a DNA octahedron, or a combination thereof. In another aspect, the folded monodispersed nucleic acid nanostructure is coated with one or more peptides selected from a poly-lysine peptide, a poly-lysine peptide having a PEG chain, a poly-aurein peptide, a poly-aurein peptide having a PEG chain, a specific cell-targeting peptide, an endosomal escape peptide, and combinations thereof. In another aspect, the endosomal escape peptide has an amino acid sequence having at least 90-95% identity to SEQ ID NO: 77-81. The composition of clause 7, wherein the endosomal escape peptide has an amino acid sequence of SEQ ID NO: 77-81. In another aspect, the endosomal escape peptide comprises a lysine10 (K10) peptide flanked by two copies of an aurein 1.2 peptide (SEQ ID NO: 77). In another aspect, the poly-lysine peptide comprises a lysine10 (K10) peptide (SEQ ID NO: 78). In another aspect, the nucleic acid nanostructure has intramolecular folding capabilities without the need for helper nucleic acid strands.

Another embodiment described herein is a method of improving cellular uptake of a nucleic acid nanostructure, the method comprising delivering to a cell a nanoparticle composition comprising a folded monodispersed nucleic acid nanostructure comprising a messenger RNA (mRNA), a single-stranded DNA (ssDNA), or a combination thereof. In one aspect, the folded monodispersed nucleic acid nanostructure is coated with one or more peptides selected from a poly-lysine peptide, a poly-lysine peptide having a PEG chain, a poly-aurein peptide, a poly-aurein peptide having a PEG chain, a specific cell-targeting peptide, an endosomal escape peptide, and combinations thereof. In another aspect, the nucleic acid nanostructure further comprises one or more DNA or RNA staple strands to promote folding and stabilization of the nucleic acid nanostructure. In another aspect, the nucleic acid nanostructure comprises a single-stranded mRNA that serves as a scaffold strand, and one or more short DNA staple strands that promote folding and stabilization of the mRNA into a compact monodispersed nanostructure. In another aspect, the nanostructure comprises mRNA with a designed single-stranded RNA to fold it into a compact nanostructure comprised of double-stranded regions, loops and cross-overs. In another aspect, the nanostructure comprises a designed single-stranded RNA where one half comprises the designed mRNA and second half comprises the designed regions to drive folding of the entire single-stranded RNA into a compact nanostructure comprised of double-stranded regions, loops and cross-overs.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a “Cargo-as-carrier” model where an mRNA or ssDNA scaffold strand is combined with a plurality of short DNA or RNA staple strands to form a folded nucleic acid nanostructure that can be further coated with various functional peptides.

FIG. 2 shows an aurein-oligolysine-aurein (aurein-K10-aurein, “AKA”) peptide coating of a small DNA nanostructure (e.g., six-helix bundle) for nuclease protection and endosomal escape.

FIG. 3A-C show designs for a DNA octahedron origami (FIG. 3A), a DNA octahedron protected mRNA (FIG. 3B), and a DNA-mRNA 6-helix bundle (FIG. 3C).

FIG. 4 shows the computational design of structured mRNA. The process takes an mRNA sequence and inserting it into a 0-crossing number RNA origami. In step 1, the mRNA sequence is identified, including the 5′ and 3′ UTR regions. In step 2, an RNA origami design is found with size greater than twice the length of the mRNA to be structured. In step 3, we use the oxView scripting interface and design tools to modify the sequence of the RNA origami design to incorporate the mRNA sequence with the 5′ UTR, the start of the coding region (39 bases), and the 3′ UTR as overhangs. This separates the origami into two strands, one containing the mRNA sequence and the other a “structuring strand” which folds the mRNA into a more compact form. Finally, oxDNA simulations of the structured mRNA and the mRNA with no structure are performed to determine the ratio of compaction.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

Described herein is the design of a DNA nanotechnology-based platform for the delivery of mRNA and/or single-stranded (ss) DNA (i.e., ssDNA) into cells. The approach relies on folding the cargo (either the mRNA or the ssDNA) into a compact, defined, monodispersed nanostructure via self-assembly with or without helper strands. Therefore, the described technology provides a novel mechanism of using nucleic acid molecules themselves as the structural material for nanoparticle formation, as opposed to packaging the nucleic acid molecules into another delivery vehicle. The nanostructure can be any compact shape (e.g., 6- or 12-helical bundles, cuboid shapes with lattice architecture, etc.).

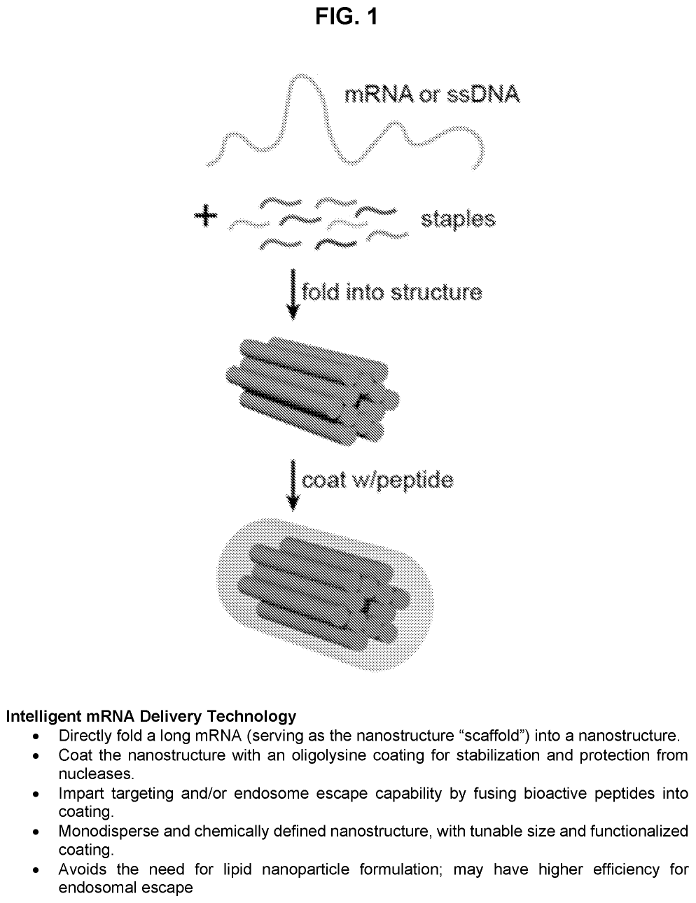

A “cargo-as-carrier” method will be developed where a nucleic acid nanostructure will be coated with peptides that protect it against degradation and impart biological activity (such as targeting or delivery into the cytosol) (FIG. 1). The functionality of these nanostructures will be probed with the delivery of mRNA or ssDNA encoding for GFP or the Cas9 protein and guide RNA, and the protection and bioactivity imparted by the peptide coating will be determined. A range of nanostructures and peptide coating designs will also be explored, with the ultimate goal of creating chemically defined, multi-functional particles for the efficient delivery of nucleic acids into cells.

This DNA nanotechnology-based cell delivery platform and approach relies on “folding” mRNA or ssDNA genes into a defined nanostructure, similar to traditional DNA origami, followed by coating the nanostructure with peptides that will provide low-salt stabilization, protection from nucleases, and endosomal-escape capabilities (FIG. 1).

These approaches will provide an alternative to packaging mRNA and ssDNA into lipid nanoparticles (e.g., the Pfizer and Moderna vaccines). By compacting nucleic acid nanostructures into a single particle, it avoids the complex formulation properties of the lipid nanoparticles. The disclosed technology also includes the use of a cationic peptide-PEG “coating” to protect the mRNA or ssDNA and help the cargo nucleic acids escape the endosome into the cytoplasm once taken up into target cells. Therefore, the proposed technology relies on folding the cargo mRNA or ssDNA into a compact and defined nanostructure, and then coating it with peptides that protect it against degradation and impart biological activity.

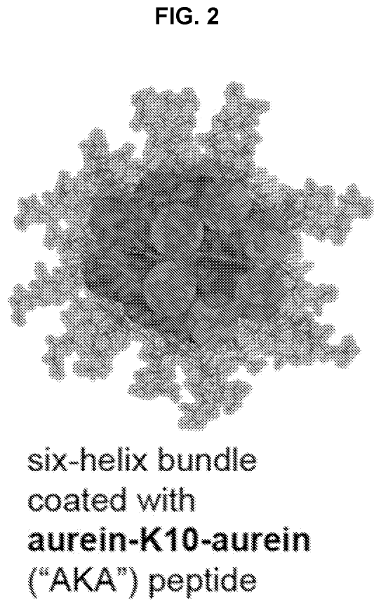

The specific mRNA strand for a gene of interest to be expressed in a target cell is produced by in vitro transcription. Alternatively, a ssDNA can be used as the cargo strand. The strand serves as the “scaffold” strand for folding into a compact nanostructure (e.g., a multi-helical bundle, or a cuboid type structure). In some embodiments, folding is achieved with the help of short DNA or RNA strand “staples”, with DNA staple strands being preferred (FIG. 1). The nanostructure can then be coated with poly-lysine (e.g., “K10”) peptides (FIG. 2), which can also bear either a PEG chain to prevent protein adsorption (e.g., protein corona formation), targeting peptides (to direct the nanoparticles to a desired cell type), or endosomal escape peptides to escape cellular endosomes and reach the cytoplasm. There is a risk that the DNA or RNA staple strands could prevent translation of the cargo mRNA, in which case these staples can be made degradable by introducing a disulfide linkage that becomes reduced in the cytoplasm, making the DNA-RNA duplex too weak to associate and encouraging the staple “fragments” to fall off and liberate the mRNA.

As described herein, the disclosed nucleic acid delivery strategy offers several unique and advantageous elements: (1) the peptide coating protects mRNA/ssDNA from degradation, and enables endosomal escape; (2) higher levels of mRNA loading are possible compared to lipid nanoparticle approaches; (3) stoichiometric and geometric control over cargo and ligands for cell targeting and barrier penetration; (4) tunable size and shape of nanoparticles; (5) potential for integration with liposome and lipid nanodisc compartments; (6) chemically defined, homogeneous nanostructures; and (7) manufacturing at a low material cost of $200 per gram using biological production, via phage, of DNA strands.

One embodiment described herein is a nanoparticle composition comprising a folded monodispersed nucleic acid nanostructure. In one aspect, the nucleic acid nanostructure comprises a messenger RNA (mRNA), a single-stranded DNA (ssDNA), or a combination thereof. In another aspect, the nucleic acid nanostructure comprises one or more nucleic acid sequences having at least 90-95% identity to SEQ ID NO: 1, 3, 5, 7, 9, 11, 13, or 15-76. In another aspect, the nucleic acid nanostructure comprises one or more nucleic acid sequences of SEQ ID NO: 1, 3, 5, 7, 9, 11, 13, or 15-76. In another aspect, the composition further comprises one or more DNA or RNA staple strands. In another aspect, the staple strands are short DNA staple strands. In another aspect, the nucleic acid nanostructure comprises a single-stranded mRNA that serves as a scaffold strand, and one or more short DNA or RNA staple strands that promote folding and stabilization of the mRNA into a compact monodispersed nanostructure. In another aspect, the folded monodispersed nucleic acid nanostructure comprises a 6-helix bundle (6HB), a 12-helix bundle, a DNA octahedron, or a combination thereof. In another aspect, the folded monodispersed nucleic acid nanostructure is coated with one or more peptides selected from a poly-lysine peptide, a poly-lysine peptide having a PEG chain, a poly-aurein peptide, a poly-aurein peptide having a PEG chain, a specific cell-targeting peptide, an endosomal escape peptide, and combinations thereof. In another aspect, the endosomal escape peptide has an amino acid sequence having at least 90-95% identity to SEQ ID NO: 77-81. The composition of clause 7, wherein the endosomal escape peptide has an amino acid sequence of SEQ ID NO: 77-81. In another aspect, the endosomal escape peptide comprises a lysine10 (K10) peptide flanked by two copies of an aurein 1.2 peptide (SEQ ID NO: 77). In another aspect, the poly-lysine peptide comprises a lysine10 (K10) peptide (SEQ ID NO: 78). In another aspect, the nucleic acid nanostructure has intramolecular folding capabilities without the need for helper nucleic acid strands.

Another embodiment described herein is a method of improving cellular uptake of a nucleic acid nanostructure, the method comprising delivering to a cell a nanoparticle composition comprising a folded monodispersed nucleic acid nanostructure comprising a messenger RNA (mRNA), a single-stranded DNA (ssDNA), or a combination thereof. In one aspect, the folded monodispersed nucleic acid nanostructure is coated with one or more peptides selected from a poly-lysine peptide, a poly-lysine peptide having a PEG chain, a poly-aurein peptide, a poly-aurein peptide having a PEG chain, a specific cell-targeting peptide, an endosomal escape peptide, and combinations thereof. In another aspect, the nucleic acid nanostructure further comprises one or more DNA or RNA staple strands to promote folding and stabilization of the nucleic acid nanostructure. In another aspect, the nucleic acid nanostructure comprises a single-stranded mRNA that serves as a scaffold strand, and one or more short DNA staple strands that promote folding and stabilization of the mRNA into a compact monodispersed nanostructure. In another aspect, the nanostructure comprises mRNA with a designed single-stranded RNA to fold it into a compact nanostructure comprised of double-stranded regions, loops and cross-overs. In another aspect, the nanostructure comprises a designed single-stranded RNA where one half comprises the designed mRNA and second half comprises the designed regions to drive folding of the entire single-stranded RNA into a compact nanostructure comprised of double-stranded regions, loops and cross-overs.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:

Clause 1. A nanoparticle composition comprising a folded monodispersed nucleic acid nanostructure.

Clause 2. The composition of clause 1, wherein the nucleic acid nanostructure comprises a messenger RNA (mRNA), a single-stranded DNA (ssDNA), or a combination thereof.

Clause 3. The composition of clause 1, wherein the nucleic acid nanostructure comprises one or more nucleic acid sequences having at least 90-95% identity to SEQ ID NO: 1, 3, 5, 7, 9, 11, 13, or 15-76.

Clause 4. The composition of clause 1, wherein the nucleic acid nanostructure comprises one or more nucleic acid sequences of SEQ ID NO: 1, 3, 5, 7, 9, 11, 13, or 15-76.

Clause 5. The composition of clause 1, further comprising one or more DNA or RNA staple strands.

Clause 6. The composition of clause 5, wherein the staple strands are short DNA staple strands.

Clause 7. The composition of clause 1, wherein the nucleic acid nanostructure comprises a single-stranded mRNA that serves as a scaffold strand, and one or more short DNA or RNA staple strands that promote folding and stabilization of the mRNA into a compact monodispersed nanostructure.

Clause 8. The composition of clause 1, wherein the folded monodispersed nucleic acid nanostructure comprises a 6-helix bundle (6HB), a 12-helix bundle, a DNA octahedron, or a combination thereof.

Clause 9. The composition of clause 1, wherein the folded monodispersed nucleic acid nanostructure is coated with one or more peptides selected from a poly-lysine peptide, a poly-lysine peptide having a PEG chain, a poly-aurein peptide, a poly-aurein peptide having a PEG chain, a specific cell-targeting peptide, an endosomal escape peptide, and combinations thereof.

Clause 10. The composition of clause 9, wherein the endosomal escape peptide has an amino acid sequence having at least 90-95% identity to SEQ ID NO: 77-81.

Clause 11. The composition of clause 9, wherein the endosomal escape peptide has an amino acid sequence of SEQ ID NO: 77-81.

Clause 12. The composition of clause 9, wherein the endosomal escape peptide comprises a lysine10 (K10) peptide flanked by two copies of an aurein 1.2 peptide (SEQ ID NO: 77).

Clause 13. The composition of clause 9, wherein the poly-lysine peptide comprises a lysine10 (K10) peptide (SEQ ID NO: 78).

Clause 14. The composition of clause 1, wherein the nucleic acid nanostructure has intramolecular folding capabilities without the need for helper nucleic acid strands.

Clause 15. A method of improving cellular uptake of a nucleic acid nanostructure, the method comprising delivering to a cell a nanoparticle composition comprising a folded monodispersed nucleic acid nanostructure comprising a messenger RNA (mRNA), a single-stranded DNA (ssDNA), or a combination thereof.

Clause 16. The method of clause 15, wherein the folded monodispersed nucleic acid nanostructure is coated with one or more peptides selected from a poly-lysine peptide, a poly-lysine peptide having a PEG chain, a poly-aurein peptide, a poly-aurein peptide having a PEG chain, a specific cell-targeting peptide, an endosomal escape peptide, and combinations thereof.

Clause 17. The method of clause 15, wherein the nucleic acid nanostructure further comprises one or more DNA or RNA staple strands to promote folding and stabilization of the nucleic acid nanostructure.

Clause 18. The method of clause 15, wherein the nucleic acid nanostructure comprises a single-stranded mRNA that serves as a scaffold strand, and one or more short DNA staple strands that promote folding and stabilization of the mRNA into a compact monodispersed nanostructure.

Clause 19. The method of clause 15, where the nanostructure comprises mRNA with a designed single-stranded RNA to fold it into a compact nanostructure comprised of double-stranded regions, loops and cross-overs.

Clause 20. The method of clause 15, where the nanostructure comprises a designed single-stranded RNA, wherein one half comprises the designed mRNA and second half comprises the designed regions to drive folding of the entire single-stranded RNA into a compact nanostructure comprised of double-stranded regions, loops and cross-overs.

### EXAMPLES

### Example 1

### “Cargo-as-Carrier” Approach

The specific mRNA and/or ssDNA for a particular gene of interest was folded into a defined shape, coated with aurein-based peptides that enable endosomal escape, and then cytoplasmic delivery into cells was assessed. The functional mRNA and/or ssDNA cargo was used as the nano-carrier itself, thereby minimizing the required number of components and eliminating the need for an additional nanoparticle carrier. The effect of shape and size of the nanostructure on the efficiency of cell delivery was evaluated by assessing uptake and gene-expression in cells.

Preliminary results have demonstrated that oligolysine peptides bearing two copies of an endosome-escape peptide (termed “aurein 1.2”) can effectively coat a small six-helix bundle DNA nanostructure (FIG. 2) and promote its release from the endosome into the cytoplasm. The 13-residue aurein 1.2 peptide (GLFDIIKKIAESF; SEQ ID NO: 77) was found to enhance endolysosomal escape and improve the cytosolic delivery of proteins it was appended to by up to ˜5-fold. In fact, this peptide can disrupt endolysosomal membranes and in such a way trigger the escape of cargo to cytosol. Importantly, aurein facilitates endolysosomal escape without concomitant disruption of the cell membrane and does not exhibit cytotoxicity.

A number of peptide variants were generated for the coating, including: K10 (SEQ ID NO 78); aurein-K10-aurein (AKA; FIG. 2; SEQ ID NO: 79); K10-PEG5k (KP) (SEQ ID NO: 80); aurein-PEG1k-K10-PEG1k-aurein (APKPA) (SEQ ID NO: 81); as well as peptides with cell-penetrating capabilities. Small nanostructures were generated with mRNA or ssDNA as the scaffold, focusing on the following as model cargo: (1) GFP; (2) Cas9+guide RNA, and (3) RNAs encoding vaccine components. Several different nanostructures were probed, which vary in shape and aspect ratio: (1) a short 6- or 12-helix bundle, as shown in FIG. 1; (2) rectangular blocks with a square-holey lattice architecture; and (3) 2D-sheet shapes. These nanostructures will be designed using caDNAno software, folded following traditional methods, and characterized by electron microscopy to verify their shape and size.

The structures were coated with the panel of peptides, which vary in the density of the bioactive signal, the length of PEG, and the molecular geometry. Crosslinking the peptide shell will also be explored to further stabilize the coating, perhaps with the addition of reductively cleavable linkages to enable coat “shedding” in the cytoplasm. To probe delivery, HEK 293 or NIH 3T3 cells will be used and uptake of the uncoated versus coated nanostructures will be evaluated, as evidenced by either GFP expression/fluorescence, or luciferase expression (for a Cas9 activity reporter). Endosomal escape will be verified by live-cell confocal microscopy, looking for co-localization of endosomal markers with fluorophores on the nanostructure. The stability of the nanostructures in cell media and to biologically relevant nuclease concentrations will also be probed. The addition of targeting peptides to the coating (through coating with two different peptides) to enhance uptake will also be explored.

### Example 2

mRNA and ssRNA Sequence Designs and DNA Origami

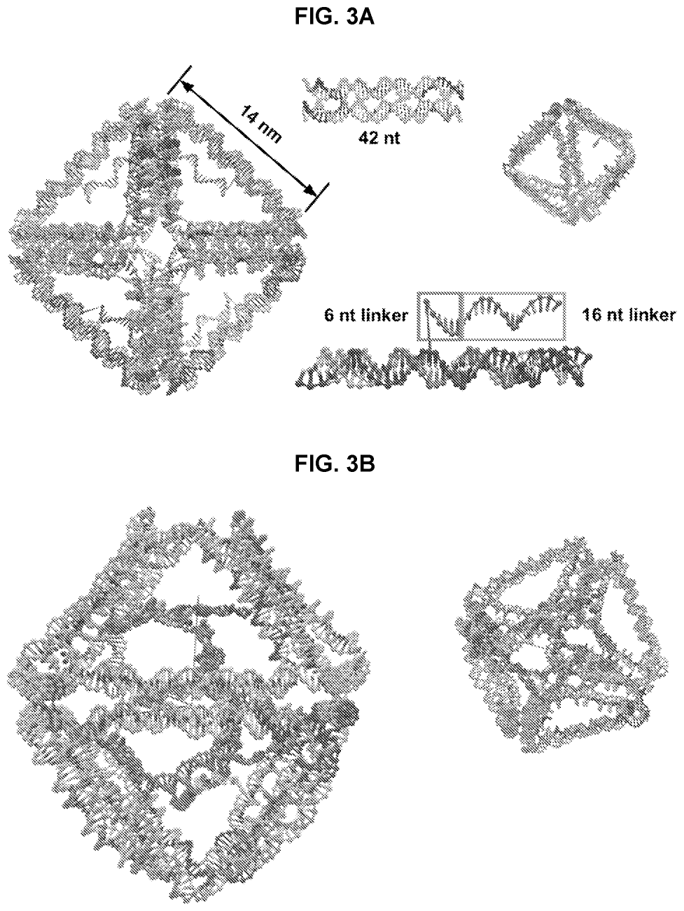

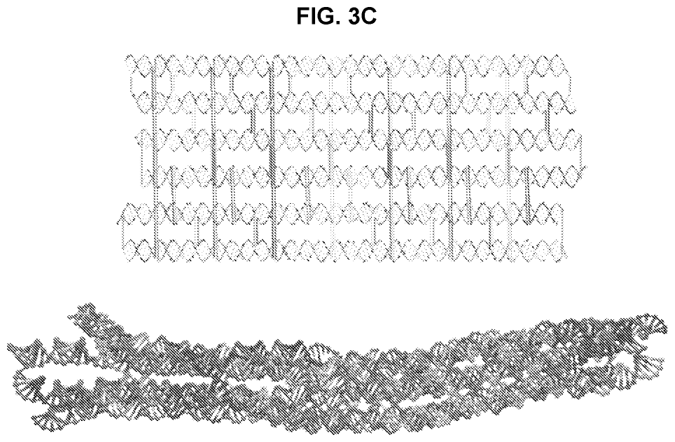

Multiple nucleic acid sequence designs will be developed for self-assembly and folding into compact nanostructures. One design includes a DNA octahedron origami-protected mRNA (FIG. 3A-B) (e.g., GFP sequence). Another design includes a DNA-mRNA (e.g., GFP sequence) hybrid six helix bundle origami (FIG. 3C).

Exemplary sequences are shown in the following tables.

TABLE 1
mRNA Folding Sequences

| Name | mRNA Sequence | Protein Sequence |
|---|---|---|
| eGFP | SEQ ID NO: 1 | SEQ ID NO: 2 |
| (TriLink) | AUGGUGAGCAAGGGCGAGGAGCUGUUCACCGGGGUGGUGCCCA | MVSKGEELFTGVVPILVELDGDVNGHK |
| | UCCUGGUCGAGCUGGACGGCGACGUAAACGGCCACAAGUUCAG | FSVSGEGEGDATYGKLTLKFICTTGKL |
| | CGUGUCCGGCGAGGGCGAGGGCGAUGCCACCUACGGCAAGCUG | PVPWPTLVTTLTYGVQCFSRYPDHMKQ |
| | ACCCUGAAGUUCAUCUGCACCACCGGCAAGCUGCCCGUGCCCU | HDFFKSAMPEGYVQERTIFFKDDGNYK |
| | GGCCCACCCUCGUGACCACCCUGACCUACGGCGUGCAGUGCUU | TRAEVKFEGDTLVNRIELKGIDFKEDG |
| | CAGCCGCUACCCCGACCACAUGAAGCAGCACGACUUCUUCAAG | NILGHKLEYNYNSHNVYIMADKQKNGI |
| | UCCGCCAUGCCCGAAGGCUACGUCCAGGAGCGCACCAUCUUCU | KVNFKIRHNIEDGSVQLADHYQQNTPI |
| | UCAAGGACGACGGCAACUACAAGACCCGCGCCGAGGUGAAGUU | GDGPVLLPDNHYLSTQSALSKDPNEKR |
| | CGAGGGCGACACCCUGGUGAACCGCAUCGAGCUGAAGGGCAUC | DHMVLLEFVTAAGITLGMDELYK |
| | GACUUCAAGGAGGACGGCAACAUCCUGGGGCACAAGCUGGAGU | |
| | ACAACUACAACAGCCACAACGUCUAUAUCAUGGCCGACAAGCA | |
| | GAAGAACGGCAUCAAGGUGAACUUCAAGAUCCGCCACAACAUC | |
| | GAGGACGGCAGCGUGCAGCUCGCCGACCACUACCAGCAGAACA | |
| | CCCCCAUCGGCGACGGCCCCGUGCUGCUGCCCGACAACCACUA | |
| | CCUGAGCACCCAGUCCGCCCUGAGCAAAGACCCCAACGAGAAG | |
| | CGCGAUCACAUGGUCCUGCUGGAGUUCGUGACCGCCGCCGGGA | |
| | UCACUCUCGGCAUGGACGAGCUGUACAAGUAA | |
| JEV-RBD | SEQ ID NO: 3 | SEQ ID NO: 4 |
| | AUGUGGCUCGUUUCCUUAGCAAUAGUGACAGCCUGCGCCGGGG | MWLVSLAIVTACAGATNLCPFGEVFNA |
| | CAACCAACCUUUGCCCCUUUGGGGAGGUAUUCAAUGCUACUAG | TRFASVYAWNRKRISNCVADYSVLYNS |
| | AUUCGCUAGCGUGUACGCUUGGAAUCGUAAGAGAAUUUCCAAU | ASFSTFKCYGVSPTKLNDLCFTNVYAD |
| | UGUGUUGCAGACUACUCAGUGCUUUAUAACUCUGCAAGCUUCU | SFVIRGDEVRQIAPGQTGKIADYNYKL |
| | CUACAUUUAAAUGUUAUGGCGUGUCACCUACAAAGCUGAAUGA | PDDFTGCVIAWNSNNLDSKVGGNYNYL |
| | UUUAUGCUUCACGAACGUCUACGCGGAUAGCUUCGUUAUCAGG | YRLFRKSNLKPFERDISTEIYQAGSTP |
| | GGGGACGAAGUGAGGCAGAUUGCCCCCGGUCAGACCGGUAAGA | CNGVEGFNCYFPLQSYGFQPTNGVGYQ |
| | UCGCAGAUUACAACUACAAGCUGCCAGACGAUUUCACUGGCUG | PYRVVVLSFELLHAPATVCGP |
| | CGUGAUAGCUUGGAAUAGCAACAACUUAGACAGCAAGGUGGGU | |
| | GGCAACUAUAACUAUCUGUACAGACUCUUUCGGAAGUCUAAUC | |
| | UUAAGCCCUUCGAAAGAGACAUAAGCACGGAGAUCUAUCAAGC | |
| | AGGGAGUACACCGUGUAAUGGUGUCGAAGGCUUCAAUUGUUAU | |
| | UUCCCUCUGCAAAGUUACGGUUUUCAACCCACCAAUGGAGUGG | |
| | GAUACCAGCCAUACAGGGUAGUUGUGUUGUCAUUUGAGCUGUU | |
| | ACACGCUCCUGCCACUGUGUGCGGGCCUUAA | |
| JEV-Spike | SEQ ID NO: 5 | SEQ ID NO: 6 |
| | AUGUGGCUGGUGAGCCUCGCCAUUGUGACGGCCUGCGCCGGGG | MWLVSLAIVTACAGAMFVFLVLLPLVS |
| | CCAUGUUUGUUUUUCUUGUUUUAUUGCCACUAGUCUCUAGUCA | SQCVNLTTRTQLPPAYTNSFTRGVYYP |
| | GUGUGUUAAUCUUACAACCAGAACUCAAUUACCCCCUGCAUAC | DKVFRSSVLHSTQDLFLPFFSNVTWFH |
| | ACUAAUUCUUUCACACGUGGUGUUUAUUACCCUGACAAAGUUU | AIHVSGTNGTKRFDNPVLPFNDGVYFA |
| | UCAGAUCCUCAGUUUUACAUUCAACUCAGGACUUGUUCUUACC | STEKSNIIRGWIFGTTLDSKTQSLLIV |
| | UUUCUUUUCCAAUGUUACUUGGUUCCAUGCUAUACAUGUCUCU | NNATNVVIKVCEFQFCNDPFLGVYYHK |
| | GGGACCAAUGGUACUAAGAGGUUUGAUAACCCUGUCCUACCAU | NNKSWMESEFRVYSSANNCTFEYVSQP |
| | UUAAUGAUGGUGUUUAUUUUGCUUCCACUGAGAAGUCUAACAU | FLMDLEGKQGNFKNLREFVFKNIDGYF |
| | AAUAAGAGGCUGGAUUUUUGGUACUACUUUAGAUUCGAAGACC | KIYSKHTPINLVRDLPQGFSALEPLVD |
| | CAGUCCCUACUUAUUGUUAAUAACGCUACUAAUGUUGUUAUUA | LPIGINITRFQTLLALHRSYLTPGDSS |
| | AAGUCUGUGAAUUUCAAUUUUGUAAUGAUCCAUUUUUGGGUGU | SGWTAGAAAYYVGYLQPRTFLLKYNEN |
| | UUAUUACCACAAAAACAACAAAAGUUGGAUGGAAAGUGAGUUC | GTITDAVDCALDPLSETKCTLKSFTVE |
| | AGAGUUUAUUCUAGUGCGAAUAAUUGCACUUUUGAAUAUGUCU | KGIYQTSNFRVQPTESIVRFPNITNLC |
| | CUCAGCCUUUUCUUAUGGACCUUGAAGGAAAACAGGGUAAUUU | PFGEVFNATRFASVYAWNRKRISNCVA |
| | CAAAAAUCUUAGGGAAUUUGUGUUUAAGAAUAUUGAUGGUUAU | DYSVLYNSASFSTFKCYGVSPTKLNDL |
| | UUUAAAAUAUAUUCUAAGCACACGCCUAUUAAUUUAGUGCGUG | CFTNVYADSFVIRGDEVRQIAPGQTGK |
| | AUCUCCCUCAGGGUUUUUCGGCUUUAGAACCAUUGGUAGAUUU | IADYNYKLPDDFTGCVIAWNSNNLDSK |
| | GCCAAUAGGUAUUAACAUCACUAGGUUUCAAACUUUACUUGCU | VGGNYNYLYRLFRKSNLKPFERDISTE |
| | UUACAUAGAAGUUAUUUGACUCCUGGUGAUUCUUCUUCAGGUU | IYQAGSTPCNGVEGFNCYFPLQSYGFQ |
| | GGACAGCUGGUGCUGCAGCUUAUUAUGUGGGUUAUCUUCAACC | PTNGVGYQPYRVVVLSFELLHAPATVC |
| | UAGGACUUUUCUAUUAAAAUAUAAUGAAAAUGGAACCAUUACA | GPKKSTNLVKNKCVNFNFNGLTGTGVL |
| | GAUGCUGUAGACUGUGCACUUGACCCUCUCUCAGAAACAAAGU | TESNKKFLPFQQFGRDIADTTDAVRDP |
| | GUACGUUGAAAUCCUUCACUGUAGAAAAAGGAAUCUAUCAAAC | QTLEILDITPCSFGGVSVITPGTNTSN |
| | UUCUAACUUUAGAGUCCAACCAACAGAAUCUAUUGUUAGAUUU | QVAVLYQDVNCTEVPVAIHADQLTPTW |
| | CCUAAUAUUACAAACUUGUGCCCUUUUGGUGAAGUUUUUAACG | RVYSTGSNVFQTRAGCLIGAEHVNNSY |
| | CCACCAGAUUUGCAUCUGUUUAUGCUUGGAACAGGAAGAGAAU | ECDIPIGAGICASYQTQTNSPGSASSV |
| | CAGCAACUGUGUUGCUGAUUAUUCUGUCCUAUAUAAUUCCGCA | ASQSIIAYTMSLGAENSVAYSNNSIAI |
| | UCAUUUUCCACUUUUAAGUGUUAUGGAGUGUCUCCUACUAAAU | PTNFTISVTTEILPVSMTKTSVDCTMY |
| | UAAAUGAUCUCUGCUUUACUAAUGUCUAUGCAGAUUCAUUUGU | ICGDSTECSNLLLQYGSFCTQLNRALT |
| | AAUUAGAGGUGAUGAAGUCAGACAAAUCGCUCCAGGGCAAACU | GIAVEQDKNTQEVFAQVKQIYKTPPIK |
| | GGAAAGAUUGCUGAUUAUAAUUAUAAAUUACCAGAUGAUUUUA | DFGGFNFSQILPDPSKPSKRSFIEDLL |
| | CAGGCUGCGUUAUAGCUUGGAAUUCUAACAAUCUUGAUUCUAA | FNKVTLADAGFIKQYGDCLGDIAARDL |
| | GGUUGGUGGUAAUUAUAAUUACCUGUAUAGAUUGUUUAGGAAG | ICAQKFNGLTVLPPLLTDEMIAQYTSA |
| | UCUAAUCUCAAACCUUUUGAGAGAGAUAUUUCAACUGAAAUCU | LLAGTITSGWTFGAGAALQIPFAMQMA |
| | AUCAGGCCGGUAGCACACCUUGUAAUGGUGUUGAAGGUUUUAA | YRFNGIGVTQNVLYENQKLIANQFNSA |
| | UUGUUACUUUCCUUUACAAUCAUAUGGUUUCCAACCCACUAAU | IGKIQDSLSSTASALGKLQDVVNQNAQ |
| | GGUGUUGGUUACCAACCAUACAGAGUAGUAGUACUUUCUUUUG | ALNTLVKQLSSNFGAISSVLNDILSRL |
| | AACUUCUACAUGCACCAGCAACUGUUUGUGGACCUAAAAAGUC | DPPEAEVQIDRLITGRLQSLQTYVTQQ |
| | UACUAAUUUGGUUAAAAACAAAUGUGUCAAUUUCAACUUCAAU | LIRAAEIRASANLAATKMSECVLGQSK |
| | GGUUUAACAGGCACAGGUGUUCUUACUGAGUCUAACAAAAAGU | RVDFCGKGYHLMSFPQSAPHGVVFLHV |
| | UUCUGCCUUUCCAACAAUUUGGCAGAGACAUUGCUGACACUAC | TYVPAQEKNFTTAPAICHDGKAHFPRE |
| | UGAUGCUGUCCGUGAUCCACAGACACUUGAGAUUCUUGACAUU | GVFVSNGTHWFVTQRNFYEPQIITTDN |
| | ACACCAUGUUCUUUUGGUGGUGUCAGUGUUAUAACACCAGGAA | TFVSGNCDVVIGIVNNTVYDPLQPELD |
| | CAAAUACUUCUAACCAGGUUGCUGUUCUUUAUCAGGAUGUUAA | SFKEELDKYFKNHTSPDVDLGDISGIN |
| | CUGCACAGAAGUCCCUGUUGCUAUUCAUGCAGAUCAACUUACU | ASVVNIQKEIDRLNEVAKNLNESLIDL |
| | CCUACUUGGCGUGUUUAUUCUACAGGUUCUAAUGUUUUUCAAA | QELGKYEQGSGYIPEAPRDGQAYVRKD |
| | CACGUGCAGGCUGUUUAAUAGGGGCUGAACAUGUCAACAACUC | GEWVLLSTFLGRSLEVLFQGPGHHHHH |
| | AUAUGAGUGUGACAUACCCAUUGGUGCAGGUAUAUGCGCUAGU | HHHSAWSHPQFEKGGGSGGGGSGGSAW |
| | UAUCAGACUCAGACUAAUUCUCCUGGCAGCGCCAGCAGUGUAG | SHPQFEK |
| | CUAGUCAAUCCAUCAUUGCCUACACUAUGUCACUUGGUGCAGA | |
| | AAAUUCAGUUGCUUACUCUAAUAACUCUAUUGCCAUACCCACA | |
| | AAUUUUACUAUUAGUGUUACCACAGAAAUUCUACCAGUGUCUA | |
| | UGACCAAGACAUCAGUAGAUUGUACAAUGUACAUUUGUGGUGA | |
| | UUCAACUGAAUGCAGCAAUCUUUUGUUGCAAUAUGGCAGUUUU | |
| | UGUACACAAUUAAACCGUGCUUUAACUGGAAUAGCUGUUGAAC | |
| | AAGACAAAAACACCCAAGAAGUUUUUGCACAAGUCAAACAAAU | |
| | UUACAAAACACCACCAAUUAAAGAUUUUGGUGGUUUUAAUUUU | |
| | UCACAAAUAUUACCAGAUCCAUCAAAACCAAGCAAGAGGUCAU | |
| | UUAUUGAAGAUCUACUUUUCAACAAAGUGACACUUGCAGAUGC | |
| | UGGCUUCAUCAAACAAUAUGGUGAUUGCCUUGGUGAUAUUGCU | |
| | GCUAGAGACCUCAUUUGUGCACAAAAGUUUAACGGCCUUACUG | |
| | UUUUGCCACCUUUGCUCACAGAUGAAAUGAUUGCUCAAUACAC | |
| | UUCUGCACUGUUAGCGGGUACAAUCACUUCUGGUUGGACCUUU | |
| | GGUGCAGGUGCUGCAUUACAAAUACCAUUUGCUAUGCAAAUGG | |
| | CUUAUAGGUUUAAUGGUAUUGGAGUUACACAGAAUGUUCUCUA | |
| | UGAGAACCAAAAAUUGAUUGCCAACCAAUUUAAUAGUGCUAUU | |
| | GGCAAAAUUCAAGACUCACUUUCUUCCACAGCAAGUGCACUUG | |
| | GAAAACUUCAAGAUGUGGUCAACCAAAAUGCACAAGCUUUAAA | |
| | CACGCUUGUUAAACAACUUAGCUCCAAUUUUGGUGCAAUUUCA | |
| | AGUGUUUUAAAUGAUAUCCUUUCACGUCUUGACCCCCCUGAGG | |
| | CUGAAGUGCAAAUUGAUAGGUUGAUCACAGGCAGACUUCAAAG | |
| | UUUGCAGACAUAUGUGACUCAACAAUUAAUUAGAGCUGCAGAA | |
| | AUCAGAGCUUCUGCUAAUCUUGCUGCUACUAAAAUGUCAGAGU | |
| | GUGUACUUGGACAAUCAAAAAGAGUUGAUUUUUGUGGAAAGGG | |
| | CUAUCAUCUUAUGUCCUUCCCUCAGUCAGCACCUCAUGGUGUA | |
| | GUCUUCUUGCAUGUGACUUAUGUCCCUGCACAAGAAAAGAACU | |
| | UCACAACUGCUCCUGCCAUUUGUCAUGAUGGAAAAGCACACUU | |
| | UCCUCGUGAAGGUGUCUUUGUUUCAAAUGGCACACACUGGUUU | |
| | GUAACACAAAGGAAUUUUUAUGAACCACAAAUCAUUACUACAG | |
| | ACAACACAUUUGUGUCUGGUAACUGUGAUGUUGUAAUAGGAAU | |
| | UGUCAACAACACAGUUUAUGAUCCUUUGCAACCUGAAUUAGAC | |
| | UCAUUCAAGGAGGAGUUAGAUAAAUAUUUUAAGAAUCAUACAU | |
| | CACCAGAUGUUGAUUUAGGUGACAUCUCUGGCAUUAAUGCUUC | |
| | AGUUGUAAACAUUCAAAAAGAAAUUGACCGCCUCAAUGAGGUU | |
| | GCCAAGAAUUUAAAUGAAUCUCUCAUCGAUCUCCAAGAACUUG | |
| | GGACGGCCAGGCCUAUGUUAGGAAGGAUGGUGAGUGGGUCCUG | |
| | GAAAGUAUGAGCAGGGGUCUGGAUACAUUCCAGAGGCUCCCAG | |
| | GGACGGCCAGGCCUAUGUUAGGAAGGAUGGUGAGUGGGUCCUG | |
| | CUGAGUACCUUCCUCGGCAGAUCCCUGGAGGUGCUGUUUCAGG | |
| | GCCCGGGGCACCACCACCAUCAUCACCAUCACAGUGCCUGGAG | |
| | UCAUCCUCAAUUCGAGAAGGGAGGCGGAAGCGGAGGGGGAGGC | |
| | UCCGGAGGAAGCGCUUGGUCCCACCCUCAAUUCGAAAAAUAA | |
| HPV-E6 | SEQ ID NO: 7 | SEQ ID NO: 8 |
| Epitope | ACAUUUGCUUCUGACACAACUGUGUUCACUAGCAACCUCAAAC | RHLDKKQRFHNIRGRWTGRCMSCCRSS |
| | AGACACCGCCGCCACCAUGCGGCACCUGGACAAGAAGCAGAGA | RTRRETQL |
| | UUCCACAACAUCAGAGGCAGAUGGACCGGCCGUUGCAUGAGCU | |
| | GCUGCAGAAGCAGCAGAACCAGAAGAGAGACACAGCUGUAGAG | |
| | CUCGCUUUCUUGCUGUCCAAUUUCUAUUAAAGGUUCCUUUGUU | |
| | CCCUAAGUCCAACUACUAAACUGGGGGAUAUUAUGAAGGGCCU | |
| | UGAGCAUCUGGAUUCUGCCUAAUAAAAAACAUUUAUUUUCAUG | |
| | CAGCAAGCUCGCUUUCUUGCUGUCCAAUUUCUAUUAAAGGUUC | |
| | CUUUGUUCCCUAAGUCCAACUACUAAACUGGGGGAUAUUAUGA | |
| | AGGGCCUUGAGCAUCUGGAUUCUGCCUAAUAAAAAACAUUUAU | |
| | UUUCAUGCAGCAAAUAAA | |
| HPV | SEQ ID NO: 9 | SEQ ID NO: 10 |
| Multi- | ACAUUUGCUUCUGACACAACUGUGUUCACUAGCAACCUCAAAC | MPKYVKQNTLKLATHGDTPTLHEYMLD |
| epitope | AGACACCaugGGGAUCCUGGGCUUCGUGUUUACCCUCGCUGCC | LQPETTTGNPCHTTKLLHRDSVDSAPI |
| | UACAUCCUGACCGCCUUCAACUCCAGCCACAAGGCUGCCUACA | LTAFNSSHKGASVTVVEGQVDYYGLYY |
| | ACACAACCCCCAUCGUGCACCUGAAGGCUGCCUACACCCUCAA | VHEGIRTYRHLDKKQRFHNIRGRWTGR |
| | GUGCCUGAGAUACAGGUUCAAAGCUGCCUACAGGCUGGAGUGC | CMSCCRSSRTRRETQLQYSNEKWTLQD |
| | GCCAUCUACUAUAAGGCUGCCUACGUGGUUGAGGGCCAGGUCG | VSLEVYLTAPTGCIKKNTTPIVHLKGD |
| | ACUACUAUGCUGCCUACAAGAGCGCCAUCGUGACACUGACCUA | ANTLKCLRYRFKKHCTLYTAVSSTWFA |
| | CGCUGCCUACCUGACCGCCCCCACAGGCUGCAUCAAGAAAGCU | FRDLCIVYRDGNPYAVCDKCLKFYSKI |
| | GCCUACACAUACACCAGCCUCAUUAUCCUGGUGUUGGCUGCCU | SGILGFVFTL |
| | ACaccauucaugauauuauucuggaaugcgugGCUGCCUACCU | |
| | GCUCAUUAGGUGUAUCAACUGCCAGAAGGCUGCCUACAUCAUU | |
| | CUGGAGUGUGUGUACUGCAAGGCUGCCUACUGCGUGUACUGUA | |
| | AGCAGCAACUCCUGAGGGCUGCCUACACAACCCUGGAGCAGCA | |
| | AUACAACAAGGCUGCCUACGCCGUGUGCGACAAAUGUCUGAAG | |
| | UUCUACGCUGCCUACGCCGAGCCCGACAGGGCUCACUACAACA | |
| | UCGUGACCUUCUGUUGCAAGGCUGCCUACGGCACCCUGGGAAU | |
| | CGUGUGUCCCAUUUGCAGCCAGAAGGCUGCCUACUUCGUGUAC | |
| | AUCCCCCUGUUUUUGAUUCACACCCAUGCCAGGUUCCUCAUAG | |
| | GACCCGGCCCUGGGAUCAGGCCCCUUCUGCUGUCCGUGAGCAC | |
| | CUACACAUCUCUCAUAAUUGGACCCGGCCCUGGGGAUAGCACA | |
| | CUCAGGCUGUGCGUGCAGUCCACCCACGUCGACAUCAGAACUC | |
| | UUUUGGAGGACUUACUGAUGGGACCCGGCCCUGGGUAUUGCUA | |
| | CGAACAGCUGAACGACUCCAGCGAGGAGGAGGAUACCGACGAA | |
| | AUCGAUuaaUCUAGAGCGGCCGCUUCGAGCCGGUUGAAUCGCU | |
| | GAUCUCACGCCGUGGUGAGCUCGCUUUCUUGCUGUCCAAUUUC | |
| | UAUUAAAGGUUCCUUUGUUCCCUAAGUCCAACUACUAAACUGG | |
| | GGGAUAUUAUGAAGGGCCUUGAGCAUCUGGAUUCUGCCUAAUA | |
| | AAAAACAUUUAUUUUCAUUGCAAAGUUCCGCGUACGUACGGCG | |
| | UC | |
| FluM1 | SEQ ID NO: 11 | SEQ ID NO: 12 |
| Epitope | ACAUUUGCUUCUGACACAACUGUGUUCACUAGCAACCUCAAAC | GILGFVFT |
| | AGACACCAUGGGGAUCCUGGGCUUCGUGUUUACCCUCUAAUCU | |
| | AGAGCGGCCGCUUCGAGCCGGUUGAAUCGCUGAUCUCACGCCG | |
| | UGGUGAGCUCGCUUUCUUGCUGUCCAAUUUCUAUUAAAGGUUC | |
| | CUUUGUUCCCUAAGUCCAACUACUAAACUGGGGGAUAUUAUGA | |
| | AGGGCCUUGAGCAUCUGGAUUCUGCCUAAUAAAAAACAUUUAU | |
| | UUUCAUUGCAAAGUUCCGCGUACGUACGGCGUC | |
| FluHA | SEQ ID NO: 13 | SEQ ID NO: 14 |
| Epitope | ACAUUUGCUUCUGACACAACUGUGUUCACUAGCAACCUCAAAC | PKYVKQNTLKLAT |
| | AGACACCGCCGCCACCAUGCCCAAGUACGUGAAGCAGAACACC | |
| | CUGAAGCUGGCCACCUAGAGCUCGCUUUCUUGCUGUCCAAUUU | |
| | CUAUUAAAGGUUCCUUUGUUCCCUAAGUCCAACUACUAAACUG | |
| | GGGGAUAUUAUGAAGGGCCUUGAGCAUCUGGAUUCUGCCUAAU | |
| | AAAAAACAUUUAUUUUCAUGCAGCAAGCUCGCUUUCUUGCUGU | |
| | CCAAUUUCUAUUAAAGGUUCCUUUGUUCCCUAAGUCCAACUAC | |
| | UAAACUGGGGGAUAUUAUGAAGGGCCUUGAGCAUCUGGAUUCU | |
| | GCCUAAUAAAAAACAUUUAUUUUCAUGCAGCAAAUAAA | |

TABLE 2
DNA-mRNA 6HB Sequences*

| | |
|---|---|
| GGGACAUUUGCUUCUGACACAACUGUGUUCACUAGCAACCUCAAACAGACACCAUGGUGAGCAAGGG | SEQ ID NO: 15 |
| CGAGGAGCUGUUCACCGGGGUGGUGCCCAUCCUGGUCGAGCUGGACGGCGACGUAAACGGCCACAAG | |
| UUCAGCGUGUCCGGCGAGGGCGAGGGCGAUGCCACCUACGGCAAGCUGACCCUGAAGUUCAUCUGCA | |
| CCACCGGCAAGCUGCCCGUGCCCUGGCCCACCCUCGUGACCACCCUGACCUAC | |
| GGGTGGGCGAACTTCATTGTGTCAGAAGCAAATGTCCC | SEQ ID NO: 16 |
| GCAAGAAATAGTAGTTCGCCGTACGTACGCGAACACAGGGGTCAG | SEQ ID NO: 17 |
| GCTAGTGGAACTTTAATATCCCCCAGTTGCGAGCTGTCACGA | SEQ ID NO: 18 |
| CTCAAGGTCAGCGACTGCACGCCGTAGGTTAGGTGGCTTGAGGTT | SEQ ID NO: 19 |
| AATAAATTGTCTGTATCGCCCTCGCCCTTGAAGCATTCAACC | SEQ ID NO: 20 |
| ACCATGGGTTTTTTATTAGGCATGGTCGGTCCTCGCACGCTGA | SEQ ID NO: 21 |
| GGCTCGAAGCGGCCGGGTCGGGGTAGCGGCCGCCGGACCCTTGCTC | SEQ ID NO: 22 |
| GAACAGCCGAGCTGGGGGTGTTCTGCTGGTAGGAATCCAGATG | SEQ ID NO: 23 |
| GGGCCGTTACAGCTAAGAAGTCGTGCTGCGCCGTTTAACCCCGGT | SEQ ID NO: 24 |
| CGTCCTCGGGCACCCGTCGCCGTCCAGCTCGATCAGCTCGATG | SEQ ID NO: 25 |
| CTTGAAGTGAAGTCGATGCCCTCCAGGATGATGTTGCAGCACG | SEQ ID NO: 26 |
| TCCTCCTTTCACCTTCAGGTAGTGGTTGTGATCCCGCCTTCG | SEQ ID NO: 27 |
| GGGCGGACTCCAGCAGATGGTGCGCTCCTCCAGGGTGTGTTGCCG | SEQ ID NO: 28 |
| TTCTTCTCCCAGGATCGCCCTCGAACTTTTGAAGAAGGACCATGTGATCGCGCTTCT | SEQ ID NO: 29 |
| TAGTTGCCGTCGTCCCACCTCGGTACTCCAGCTTGTGCGCTTGTCTTGCTCA | SEQ ID NO: 30 |
| TGTTGTAGTTGCGCGGGTCTTG | SEQ ID NO: 31 |
| AAAAAAAAGAGGACTTAGGGAACAAAGGAACC | SEQ ID NO: 32 |
| CTTGCCGCAGGGTGCACCACGGCGTGAGACCCTTCATGCAATGAA | SEQ ID NO: 33 |
| ACTTGTGTTCATGTCTCTAGAATTACTTGCGCCGATGCACGCTGC | SEQ ID NO: 34 |
| GGCATGGCGGACTTGCGTCCATGCCGAGAGTCGGGCAGTGGCGGAT | SEQ ID NO: 35 |
| CGGTTCAGGACGTAGGCGGCGGTCACGAACTGGGTGCTGATGCCG | SEQ ID NO: 36 |
| CGTTGGGGTCTGGCCATGATATAGACGTTGTGGC | SEQ ID NO: 37 |
| TTTAATAGAAACGGGCAGCTTG | SEQ ID NO: 38 |
| CCGGTGGTGCAGATCAGGGCATTGGACA | SEQ ID NO: 39 |

*None of the mRNA sequences listed above include the polyA tail; assume lengths of 100-200 A′s at the 3′-end.

TABLE 3
DNA Octahedron Sequences

| | |
|---|---|
| AATGCTACTACTATTAGTAGAATTGATGCCACCTTTTCAGCTCGCGCCCCAAATGAAAATATAGCTAA | SEQ ID NO: 40 |
| ACAGGTTATTGACCATTTGCGAAATGTATCTAATGGTCAAACTAAATCTACTCGTTCGCAGAATTGGG | |
| AATCAACTGTTATATGGAATGAAACTTCCAGACACCGTACTTTAGTTGCATATTTAAAACATGTTGAG | |
| CTACAGCATTATATTCAGCAATTAAGCTCTAAGCCATCCGCAAAAATGACCTCTTATCAAAAGGAGCA | |
| ATTAAAGGTACTCTCTAATCCTGACCTGTTGGAGTTTGCTTCCGGTCTGGTTCGCTTTGAAGCTCGAA | |
| TTAAAACGCGATATTTGAAGTCTTTCGGGCTTCCTCTTAATCTTTTTGATGCAATCCGCTTTGCTTCT | |
| GACTATAATAGTCAGGGTAAAGACCTGATTTTTGATTTATGGTCATTCTCGTTTTCTGAACTGTTTAA | |
| AGCATTTGAGGGGGATTCAATGAATATTTATGACGATTCCGCAGTATTGGACGCTATCCAGTCTAAAC | |
| ATTTTACTATTACCCCCTCTGGCAAAACTTCTTTTGCAAAAGCCTCTCGCTATTTTGGTTTTTATCGT | |
| CGTCTGGTAAACGAGGGTTATGATAGTGTTGCTCTTACTATGCCTCGTAATTCCTTTTGGCGTTATGT | |
| TTCCGTTAGTTCGTTTTATTAACGTAGATTTTTCTTCCCAACGTCCTGACTGGTATAATGAGCCAGTT | |
| ATCTGCATTAGTTGAATGTGGTATTCCTAAATCTCAACTGATGAATCTTTCTACCTGTAATAATGTTG | |
| TTCCGTTAGTTCGTTTTATTAACGTAGATTTTTCTTCCCAACGTCCTGACTGGTATAATGAGCCAGTT | |
| CTTAAAATCGCATAAGGTAATTCACAATGATTAAAGTTGAAATTAAACCATCTCAAGCCCAATTTACT | |
| ACTCGTTCTGGTGTTTCTCGTCAGGGCAAGCCTTATTCACTGAATGAGCAGCTTTGTTACGTTGATTT | |
| GGGTAATGAATATCCGGTTCTTGTCAAGATTACTCTTGATGAAGGTCAGCCAGCCT | |
| CAAATGGTCAATTTTTTAACCTGTTTAGGTGGCATCATTTTTATTCTACTAA | SEQ ID NO: 41 |
| TTTTCATTTCTCATAGCTGGGGGCGCGAGCTGAAAAGCTATA | SEQ ID NO: 42 |
| TTAAATATGCATTTTTACTAAAGTACGATTCCCAATTTTTTTCTGCGAACGA | SEQ ID NO: 43 |
| CATATAACAGTTGGTGTCTGGAAGTTTCTACCTGAGAGATTC | SEQ ID NO: 44 |
| TAGAGCTTAATTTTTTTGCTGAATATTGACCTTCATCTTTTTAAGAGTAATC | SEQ ID NO: 45 |
| TTAATTGCTCCTTTTTTTTTGATAAGGCATCAAAAAGTTTTTATTAAGAGGA | SEQ ID NO: 46 |
| ACTTCAAATATTTTTTCGCGTTTTAAAACTCCAACAGTTTTTGTCAGGATTA | SEQ ID NO: 47 |
| AAGAGCCCATCCCCCTCAAATGCTCATAAATATTCATTGAGA | SEQ ID NO: 48 |
| TAACGCCAAAATTTTTGGAATTACGAAGGTCTTTACCTTTTTCTGACTATTA | SEQ ID NO: 49 |
| CGTCCAATACTTTTTTGCGGAATCGTTTAAACAGTTCTTTTTAGAAAACGAG | SEQ ID NO: 50 |
| TCATAACCCTCTTTTTGTTTACCAGATGCAAAAGAAGTTTTTTTTTGCCAGA | SEQ ID NO: 51 |
| ATCAAGATAATAGCGAGAGGCTTTCGACGATAAAAACCAATC | SEQ ID NO: 52 |
| AGTTGAGATTTTTTTTAGGAATACCATTACCCAAATCTTTTTAACGTAACAA | SEQ ID NO: 53 |
| TCAGTGAATAATTTTTGGCTTGCCCTAACAACATTATTTTTTTACAGGTAGA | SEQ ID NO: 54 |
| TTTTAAGAACTTTTTTGGCTCATTATACGTTGGGAAGTTTTTAAAAATCTAC | SEQ ID NO: 55 |
| TAGTAAATTGGTTTTTGCTTGAGATGAACTTTAATCATTTTTTTGTGAATTA | SEQ ID NO: 56 |
| TTGCGGATGGCTTAGTCAGAAGCAAAGCGGATTAGGTCATTTGTTGTTTTACTTGTACAGCTCG | SEQ ID NO: 57 |
| TAAATCAAAAATCGGCATAGTAAGAGCAACACTAAATGACCAGTTGTTCAGGGTGTCGCCCTCG | SEQ ID NO: 58 |
| TAATGCAGATACATTGACAAGAACCGGATATTCACATTCAACGTTGTTCACGCTGAACTTGTGG | SEQ ID NO: 59 |
| GCATTAGGCTGGCAATGCTGTAGCTCAACATGTTTAGTAGTAGTTGTTGTTGTGGCTGTTGTAC | SEQ ID NO: 60 |
| TAGTAAAATCAGGACCAGTGTTTAGACTGGATAGGGGGGTAAGTTGTTGGGCATGGCGGACTTG | SEQ ID NO: 61 |
| CACCAGAACGAGGTTAATAAAACGAACTAACGGGACGAGAAAGTTGTTGGTGGGCCAGGGCACG | SEQ ID NO: 62 |
| AGTTTGACATTTCGTTTACATTAGATACATTTCGGTAGATTTGTTGTTGGCGAGCTGCACGCTG | SEQ ID NO: 63 |
| CAAAGCGTGCGACCTTAAACCAGACCGGAAGCATTCGAGCTTGTTGTTTTTGCTCAGGGCGGAC | SEQ ID NO: 64 |

### Example 3

mRNA for Packaging as a Single-Stranded RNA

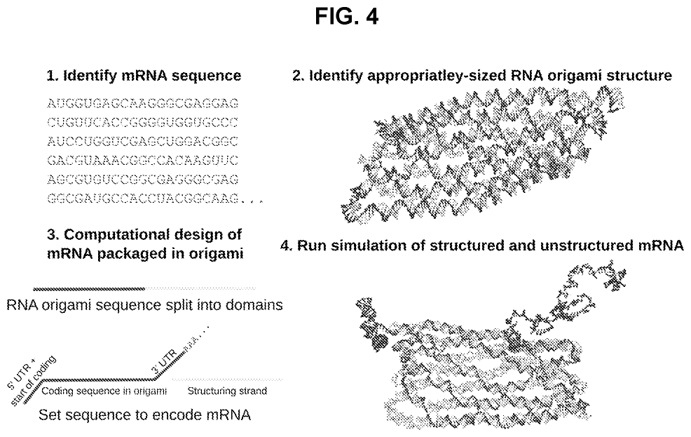

Another approach described herein for mRNA delivery relies on designing the RNA single-stranded sequence to include the mRNA as well as a designed section so that the whole sequence folds into a compact nanostructure. The process relies on using the desired mRNA sequence (along with its 5′ and 3′ UTR regions) in the first half of the designed sequence and designing the second half so that the entire RNA sequence folds into a single compact structure, called single-stranded RNA origami. We use a computational design pipeline, including coarse-grained simulation with our software oxRNA, to design and assess the folded RNA structure. The process is illustrated in FIG. 4, along with an example from a simulation of the folded structure.

The resulting structure is more compact, allowing for higher number of RNAs to be delivered together, as well as includes more double-stranded regions, thus slowing-down RNA degradation.

The examples of designed sequences designed to fold into target compact shapes are listed in Table 4 below. It includes full designed ssRNA sequences that include mRNA and the region that drives the folding into compact nanostructure (the complementary structuring strand).

TABLE 4
Single-stranded RNA Packaging Sequences

| | mRNA sequence | Complementary structuring |
|---|---|---|
| Protein encoded | (with 5′ and 3′ UTRs)* | strand |
| eGFP | SEQ ID NO: 65 | SEQ ID NO: 66 |
| | ACAUUUGCUUCUGACACAACUGUGUUCACUAGC | UAAGUAGAAGUUGCCAUCGUAGUCGCACGA |
| | AACCUCAAACAGACACCAUGGUGAGCAAGGGCG | CCUACUUAUGACGAACUUCGGUUAAGUGGC |
| | AGGAGCUGUUCACCGGGGUGGUGCCCAUCCUGG | UGACGUCCUAACAGUGCGUGCAAAAAAGAC |
| | UCGAGCUGGACGGCGACGUAAACGGCCACAAGU | CUACGAAGCCAGAGUUCGUUCCAGUGUGAA |
| | UCAGCGUGUCCGGCGAGGGCGAGGGCGAUGCCA | AGUGCACAUCACGAGUUGUGCCAAUGCACG |
| | CCUACGGCAAGCUGACCCUGAAGUUCAUCUGCA | UUGCAUCGAGAGUUAAUCCCGUCUUAAGUA |
| | CCACCGGCAAGCUGCCCGUGCCCUGGCCCACCC | GCAAGGCACCUGAAUGGAAGUUGAUUCGUC |
| | UCGUGACCACCCUGACCUACGGCGUGCAGUGCU | UAGCAAUAGACGAAUCAUGCUGAUCUCAGG |
| | UCAGCCGCUACCCCGACCACAUGAAGCAGCACG | UGCUCACUUGAUUAAGACGGCUGUUUAUCU |
| | ACUUCUUCAAGUCCGCCAUGCCCGAAGGCUACG | CGAUGCUCGCCCUCUUGGCACAAUCGAACU |
| | UCCAGGAGCGCACCAUCUUCUUCAAGGACGACG | UGUGCACUUCAGCACGGGAACGAACUCUGG |
| | GCAACUACAAGACCCGCGCCGAGGUGAAGUUCG | CUUCGUAGGUCAAAAAAGCACGCAAGCAUG |
| | AGGGCGACACCCUGGUGAACCGCAUCGAGCUGA | UAACGUCAGCCUAACGCUUGAAGUUCGCAG |
| | AGGGCAUCGACUUCAAGGAGGACGGCAACAUCC | GUGUGAGGUCGUGCAUGUGGUCUGGCAACU |
| | UGGGGCACAAGCUGGAGUACAACUACAACAGCC | CUGCUUGUUUACUUGUACAGCUCGUCCAUG |
| | ACAACGUCUAUAUCAUGGCCGACAAGCAGAAGA | CCGGAUCAGCACCCGGCGAAAAAAACGAAC |
| | ACGGCAUCAAGGUGAACUUCAAGAUCCGCCACA | UGUGAGGUAACCAUGUGGAUACUCUUCUCG |
| | ACAUCGAGGACGGCAGCGUGCAGCUCGCCGACC | UUGGCUUGCCGUCUCAGGGCUCACCAGGUG |
| | ACUACCAGCAGAACACCCCCAUCGGCGACGGCC | CUCAGGUAGUGGUUGUCGGGCAGUCACACU |
| | CCGUGCUGCUGCCCGACAACCACUACCUGAGCA | GGGCCGUCGAUACAUGCUGUGUUUCAAAAA |
| | CCCAGUCCGCCCUGAGCAAAGACCCCAACGAGA | ACGUGGUCGCACUAUAGCACGCUGCCUCGU |
| | AGCGCGAUCACAUGGUCCUGCUGGAGUUCGUGA | ACAAUGUAGCGAGAAGUCGUGAAGUUCACC |
| | CCGCCGCCGGGAUCACUCUCGGCAUGGACGAGC | UUGAUGCCGUUCUUUCUACUUACGGCCAUG |
| | UGUACAAGUAAGGGACAUUUGCUUCUGACACAA | AUCAAGUGAUUGUGGCUUACCUCACUGUAC |
| | CUGUGUUCACUAGCAACCUCAAACAGACACC | UGAAAAAAGUGCCCCAAGAUUUGGCCGUCC |
| | o(ply-A150) | UAUGAACUUUCGAUGCCCUUCAGCUCGAUG |
| | | CGGUGGACUGGGGUGUCGCCCCUCGUGAUC |
| | | ACCUCGGAAGCGUUAUUGUAGUUGUAUAGU |
| | | GCCUUGAAGAAAAAAGUGCGCUCUUGAUCA |
| | | UAGCCUUCGGGCAUGGCGGACUUGAGCGGA |
| | | UCUUGCUGCUUCGACUACGAGGGGUAGCAU |
| | | AAACAGCACUGCACGAGAGUAUCCAGGGUG |
| | | GCAAAUCUUGUGGGCCAAAAAACGGGCAGC |
| | | UUGCCGGUGGUGCAGCCUUGAAGCAGGGUC |
| | | AGGGUCUUUGAGGUGGCAAACGUGCAGCCC |
| | | UCGCCCACACCUGUGAACUUGUGUACGAGU |
| | | ACGUCGCCUGAUCAAGCGACCAGGAUGGG |
| JEV-RBD | SEQ ID NO: 67 | SEQ ID NO: 68 |
| | GGGACAUUUGCUUCUGACACAACUGUGUUCACU | GGUCAUCGCCUCAAACGUUAGGUGUCACAU |
| | AGCAACCUCAAACAGACACCAUGUGGCUCGUUU | UGUGGAAUCGCAAAAAACAUACCGACUUCC |
| | CCUUAGCAAUAGUGACAGCCUGCGCCGGGGCAA | AUUAUGGGACACGUCGCUUAUUCUUGGUAA |
| | CCAACCUUUGCCCCUUUGGGGAGGUAUUCAAUG | GUAGAAGUUGCCAUCGUAGUCGCACGACCU |
| | CUACUAGAUUCGCUAGCGUGUACGCUUGGAAUC | ACUUAUGACGAACUUCGGUUAAGUGGCUGA |
| | GUAAGAGAAUUUCCAAUUGUGUUGCAGACUACU | CGUGCUAACAGUGCGUGCAAAAAAGACCUA |
| | CAGUGCUUUAUAACUCUGCAAGCUUCUCUACAU | CGAAGCCAGAGUUCGUUCCAGUGUGAAAGU |
| | UUAAAUGUUAUGGCGUGUCACCUACAAAGCUGA | GCACAUCACGAGUUGUGCCAAUGCACGUUG |
| | AUGAUUUAUGCUUCACGAACGUCUACGCGGAUA | CAUCGAGAGUUAAUCCCGUCUUAAGUAGCA |
| | GCUUCGUUAUCAGGGGGGACGAAGUGAGGCAGA | AGGCACCUGAAUGGAAGUUGAUUCGUCUAG |
| | UUGCCCCCGGUCAGACCGGUAAGAUCGCAGAUU | AAAUAGACGAAUCAUGCUGAUCUCAGGUGC |
| | ACAACUACAAGCUGCCAGACGAUUUCACUGGCU | UCACUUGAUUAAGACGGCUGUUUAUCUCGA |
| | GCGUGAUAGCUUGGAAUAGCAACAACUUAGACA | UGCUUCCGCUUGGCACAACGCGCCGUGCAC |
| | GCAAGGUGGGUGGCAACUAUAACUAUCUGUACA | UUCCCUGAACGAACUCUGGCUUCGUAGGUC |
| | GACUCUUUCGGAAGUCUAAUCUUAAGCCCUUCG | AAAAAAGCACGCAAGCAUGUAACGUCAGCC |
| | AAAGAGACAUAAGCACGGAGAUCUAUCAAGCAG | UAACGCUUGAAGUUCGCAGGUGUGAGGUCG |
| | GGAGUACACCGUGUAAUGGUGUCGAAGGCUUCA | UGCUGCGUUUGGCAACUUGCUUGUCCAAGA |
| | AUUGUUAUUUCCCUCUGCAAAGUUACGGUUUUC | AUAAGCGACGUGUCCCAUAGAUCAGCACGG |
| | AACCCACCAAUGGAGUGGGAUACCAGCCAUACA | UAUGAAAAAAGCGAUUCGUGAGGUAGACAC |
| | GGGUAGUUGUGUUGUCAUUUGAGCUGUUACACG | CUAGAUACUCUGGCGAUGACGGUUCCCGCC |
| | CUCCUGCCACUGUGUGCGGGCCUUAAUUCUAGA | CUGCUUUCGGCGUGUCGCUCUGCUCACACU |
| | GCGGCCGCUUCGAGCCGGUUGAAUCGCUGAUCU | GCCCUGUUAUACAUGCUCCCUUCAAAAAAC |
| | CACGCCGUGGUGAGCUCGCUUUCUUGCUGUCCA | GGGUUGGCACUAUACUUUGCCUCGUACAUU |
| | AUUUCUAUUAAAGGUUCCUUUGUUCCCUAAGUC | ACUUCGGCGCCCUUCCGGUGUCUCCCUCUA |
| | CAACUACUAAACUGGGGGAUAUUAUGAAGGGCC | CUUAGUCUCCGUCAAGUGAUCUCUUUCUAC |
| | UUGAGCAUCUGGAUUCUGCCUAAUAAAAAACAU | CUCACUGUGAAAAAACCGGAAGAUUUGCGU |
| | UUAUUUUCAUUGCAAAGUUCCGCGUACGUACGG | GUUGUCUGCCCCCCCCUUGCUGUCUGUUGU |
| | CGUC (poly-A150) | GUGGCGCGCUUCCUCGUGAUGUGUAAGCGU |
| | | UAGCUUGUGUAUAGUGCUGCGUCAAAAAAG |
| | | UCUGCCUUGAUCAUCUGCCUCCUUCGUCCC |
| | | CCCUGGGCCUUCUUCCGCGGACUACGACGU |
| | | GGCAUAAACAGUUCGCUUUAGAGUAUCCCG |
| | | CCUCAAAUCUUUGUGAAAAAACUGCGGUUU |
| | | GCCUGGUUGUUGCCUUCUUGGCUCUUCGAA |
| | | CGUGCAGUCCGCUCACACCUGUGUGCUGUA |
| | | CGAGCUCCCCUGAUCAAGGGUUGGUUGCCC |
| | | C |
| HPV-E6 Epitope | SEQ ID NO: 69 | SEQ ID NO: 70 |
| | ACAUUUGCUUCUGACACAACUGUGUUCACUAGC | AAUGAGUCAGGACUUUGUAGUCGGAGUCGG |
| | AACCUCAAACAGACACCACAUUUGCUUCUGACA | AAAAAACACCAGUCACAAUGUAUCGUACGC |
| | CAACUGUGUUCACUAGCAACCUCAAACAGACAC | UUGCUACUAGGAGCUCGUCAUGACGUUGAG |
| | CGCCGCCACCAUGCGGCACCUGGACAAGAAGCA | AGCCUGUUAACUAGACACGUUCCUAAGGGU |
| | GAGAUUCCACAACAUCAGAGGCAGAUGGACCGG | UAGCCACACAUUAAUAUCGGGCCUGACACA |
| | CCGUUGCAUGAGCUGCUGCAGAAGCAGCAGAAC | GGACACGAAAAAAGAAGGUGCUGUUAGUUG |
| | CAGAAGAGAGACACAGCUGUAGAGCUCGCUUUC | GACAGGUACUAUCAUCUCAAGUCGAUAGUC |
| | UUGCUGUCCAAUUUCUAUUAAAGGUUCCUUUGU | CAAGUAGGUUUGAACCAUGCAUAGCUUGUA |
| | UCCCUAAGUCCAACUACUAAACUGGGGGAUAUU | UCAGGUCAUCGCCUCAAACGUUAGGUGUCA |
| | AUGAAGGGCCUUGAGCAUCUGGAUUCUGCCUAA | CAUUGUGGAAUCGCAAAAAACAUACCGACU |
| | UAAAAAACAUUUAUUUUCAUGCAGCAAGCUCGC | UCCAUUAUGGGACACGUCGCUUAUUCUUGG |
| | UUUCUUGCUGUCCAAUUUCUAUUAAAGGUUCCU | UAAGUAGAAGUUGCCAUCGUAGUCGCACGA |
| | UUGUUCCCUAAGUCCAACUACUAAACUGGGGGA | CCUACUUAUGACGAACUUCGGUUAAGUGGC |
| | UAUUAUGAAGGGCCUUGAGCAUCUGGAUUCUGC | UGACGUACUAACAGUGCGUGCAAAAAAGAC |
| | CUAAUAAAAAACAUUUAUUUUCAUGCAGCAAAU | CUACGAAGCCAGAGUUCGUUCCAGUGUGAA |
| | AAAAGCUCGCUUUCUUGCUGUCCAAUUUCUAUU | AGUGCACAUCACGAGUUGUGCCAAUGCACG |
| | AAAGGUUCCUUUGUUCCCUAAGUCCAACUACUA | UUGCAUCGAGAGUUAAUCCCGUCUUAAGUA |
| | AACUGGGGGAUAUUAUGAAGGGCCUUGAGCAUC | GCAAGGCACCUGAAUGGAAGUUGAUUCGUC |
| | UGGAUUCUGCCUAAUAAAAAACAUUUAUUUUCA | UAGAAAUAGACGAAUCAUGCUGAUCUCAGG |
| | U (poly-A150) | UGCUCACUUGAUUAAGACGGCUGUUUAUCU |
| | | CGAUGCCGCUGUUUGGCACAACCGUGCGUG |
| | | CACUUAUGAUAGUGAACGAACUCUGGCUUC |
| | | GUAGGUCAAAAAAGCACGCAAGCAUGUAAC |
| | | GUCAGCCUAACGCUUGAAGUUCGCAGGUGU |
| | | GAGGUCGUGCUUGUUGGCAACUGUCAUGAC |
| | | CCAAGAAUAAGCGACGUGUCCCAUAGAUCA |
| | | GCACGGUAUGAAAAAAGCGAUUCGUGAGGU |
| | | AGACACCUAGAUACUCUGGCGAUGACCGGA |
| | | GCUAUGCUUUUUUUAACCUACUUGGACUAU |
| | | CGACUUGAGUCACACUGACCUGUCCAUACA |
| | | UGCUCACCUUCAAAAAACGUGUCCGCACUA |
| | | UAGCCCGAUAUCUCGUACAGGCUAACCGGC |
| | | UUGCCGUGUCUAGUUAACAGGCUCUCAACU |
| | | CUACUUAGAGCUCCUAUCAAGUGACGUACG |
| | | AUUACCUCACACUGGUGAAAAAACCGACUC |
| | | AAGAUUUGAAGUCCUGUUGUUUUUGCUGCU |
| | | GUUGUAUGGUUCAGGCGUCUCGUGAUUCGG |
| | | CCAAGCGUUAUUCCCCCUAUAGUGCGUUGG |
| | | CAAAAAACGCUUGAUCAUGUUGGCGCGGCC |
| | | UUAGGAAUGCUGGACUACGAUUUUUUCAUA |
| | | AACAGUCCGUGCAGAGUAUCCUUCUCAAAU |
| | | CUUGUUUGAAAAAACCUUGGGCGGCCUUAC |
| | | UCAUUUUUGGCGCUGAUACACGGCUCUAAC |
| | | GUGCAGUCUCUCUUCACACCUGGCUGCUUC |
| | | UGUACGAGCUCUGCUGAUCAAGCCUCUGCC |
| | | UCUG |
| HPV Multi-epitope | SEQ ID NO: 71 | SEQ ID NO: 72 |
| | ACAUUUGCUUCUGACACAACUGUGUUCACUAGC | GGGACAUUUGCUUCUGACACAACUGUGUUC |
| | AACCUCAAACAGACACCAUGCGGGUGACCGCCC | ACUAGCAACCUCAAACAGACACCAUGGGGA |
| | CCCGGACCCUGAUCCUGCUGCUGAGCGGCGCCC | UCCUGGGCUUCGUGUUUACCCUCGCUGCCU |
| | UGGCCCUGACCGAAACCUGGGCCGGCAGCGGCG | ACAUCCUGACCGCCUUCAACUCCAGCCACA |
| | GCAGCGGCGGCGGCAGCGGUGGACCCAAGUACG | AGGCUGCCUACAACACAACCCCCAUCGUGC |
| | UGAAGCAGAACACCCUGAAGCUGGCCACCCACG | ACCUGAAGGCUGCCUACACCCUCAAGUGCC |
| | GCGACACCCCUACCCUGCACGAGUACAUGCUGG | UGAGAUACAGGUUCAAAGCUGCCUACAGGC |
| | ACCUGCAGCCUGAGACAACCACCGGCAACCCCU | UGGAGUGCGCCAUCUACUAUAAGGCUGCCU |
| | GCCACACCACCAAGCUGCUGCACAGAGAUAGCG | ACGUGGUUGAGGGCCAGGUCGACUACUAUG |
| | UGGACAGCGCCCCUAUCCUGACCGCCUUCAACA | CUGCCUACAAGAGCGCCAUCGUGACACUGA |
| | GCAGCCACAAGGGCGCCAGCGUGACCGUGGUGG | CCUACGCUGCCUACCUGACCGCCCCCACAG |
| | AAGGCCAGGUGGACUACUACGGCCUGUACUACG | GCUGCAUCAAGAAAGCUGCCUACACAUACA |
| | UGCACGAGGGCAUCAGAACCUACCGGCACCUGG | CCAGCCUCAUUAUCCUGGUGUUGGCUGCCU |
| | ACAAGAAGCAGAGAUUCCACAACAUCAGAGGCA | ACACCAUUCAUGAUAUUAUUCUGGAAUGCG |
| | GAUGGACCGGCCGUUGCAUGAGCUGCUGCAGAA | UGGCUGCCUACCUGCUCAUUAGGUGUAUCA |
| | GCAGCAGAACCAGAAGAGAGACACAGCUGCAAU | ACUGCCAGAAGGCUGCCUACAUCAUUCUGG |
| | ACAGCAACGAGAAGUGGACCCUGCAGGACGUGU | AGUGUGUGUACUGCAAGGCUGCCUACUGCG |
| | CCCUGGAAGUGUACCUGACCGCCCCUACCGGCU | UGUACUGUAAGCAGCAACUCCUGAGGGCUG |
| | GCAUCAAGAAGAAUACCACCCCUAUCGUGCACC | CCUACACAACCCUGGAGCAGCAAUACAACA |
| | UGAAAGGCGACGCCAACACCCUGAAGUGCCUGA | AGGCUGCCUACGCCGUGUGCGACAAAUGUC |
| | GAUACAGAUUCAAGAAGCACUGCACCCUGUACA | UGAAGUUCUACGCUGCCUACGCCGAGCCCG |
| | CCGCCGUGUCCAGCACCUGGUUCGCCUUCAGAG | ACAGGGCUCACUACAACAUCGUGACCUUCU |
| | AUCUGUGCAUCGUGUACCGGGACGGCAACCCCU | GUUGCAAGGCUGCCUACGGCACCCUGGGAA |
| | ACGCCGUGUGCGACAAGUGCCUGAAGUUCUACA | UCGUGUGUCCCAUUUGCAGCCAGAAGGCUG |
| | GCAAGAUCAGCGGCAUCCUGGGCUUCGUGUUCA | CCUACUUCGUGUACAUCCCCCUGUUUUUGA |
| | CCCUGGGCGGCAGCCUGGGCGGCGGCGGCAGCG | UUCACACCCAUGCCAGGUUCCUCAUAGGAC |
| | GCAUCGUGGGCAUCGUGGCCGGCCUGGCCGUGC | CCGGCCCUGGGAUCAGGCCCCUUCUGCUGU |
| | UGGCCGUGGUGGUGAUCGGCGCCGUGGUGGCCA | CCGUGAGCACCUACACAUCUCUCAUAAUUG |
| | CCGUGAUGUGCCGGCGGAAAAGCAGCGGCGGCA | GACCCGGCCCUGGGGAUAGCACACUCAGGC |
| | AGGGCGGCAGCUACAGCCAGGCCGCCAGCAGCG | UGUGCGUGCAGUCCACCCACGUCGACAUCA |
| | ACAGCGCCCAGGGCAGCGACGUGAGCCUGACCG | GAACUCUUUUGGAGGACUUACUGAUGGGAC |
| | CCUGAAGCUCGCUUUCUUGCUGUCCAAUUUCUA | CCGGCCCUGGGUAUUGCUACGAACAGCUGA |
| | UUAAAGGUUCCUUUGUUCCCUAAGUCCAACUAC | ACGACUCCAGCGAGGAGGAGGAUACCGACG |
| | UAAACUGGGGGAUAUUAUGAAGGGCCUUGAGCA | AAAUCGAUUAAGAGUUGUGCCAAUGCACGU |
| | UCUGGAUUCUGCCUAAUAAAAAACAUUUAUUUU | UGCAUCGAGAUGGCGCUCCGUCUUAAGCUC |
| | CAUGCAGCAAGCUCGCUUUCUUGCUGUCCAAUU | CAAGGCACCUGAAUGUGUAGUGAUUCGUCU |
| | UCUAUUAAAGGUUCCUUUGUUCCCUAAGUCCAA | AGAAAUAGACGAAUCAUGCUGAUCUCAGGU |
| | CUACUAAACUGGGGGAUAUUAUGAAGGGCCUUG | GCUCACUUGAUUAAGACGGCUGUUUAUCUC |
| | AGCAUCUGGAUUCUGCCUAAUAAAAAACAUUUA | GAUGCGCAGCCUUUUGGCACAAGGUAGGCA |
| | UUUUCAUGCAGCAAAUAAA (poly-A150) | GGAUUUCUUGGGACACGUCCUCCUCGCUGG |
| | | AGUCGUUCAGCCUGACAAUGAAAGAGGAUU |
| | | CUCCGGGUAAGCAUGUAAAGUCCUCCUAAC |
| | | GCUUGCUGAUGGCAGGUGUGAUGGACUGCG |
| | | UAGGUCAUCUGAGUUAGCCUUGUCCAGGGC |
| | | CGGGUCCAAUUAUGAGAAGAUCAGCACUGC |
| | | UCACAUCACUAUGAAAGGCGAUAGGCCCAG |
| | | GCGUGAGGUAGAUGAGGAGAUACUCUGGGG |
| | | UGUGCGCACUUGAAAGGGGGCGAUACACCA |
| | | AGUAGGCAGCCUUCUGGCUGCAAGGCGGUA |
| | | UCACGAUUCCAUACAUGCUCUAGGCACAAC |
| | | GCAUGGAAAUUCACGCGUUGUUGUCGCACU |
| | | AUAGGUCGGGCUCUCGUACACGAGCGUCGC |
| | | GGUCAGCACAUUUGUCGCACACGGCGUAGG |
| | | CUGCUAUCCGGUAUUGCAUCAAGUGACUUG |
| | | UGUUUACCUCACACAGGAGAAUGUCCUAGA |
| | | AAUCCCCUAAAGAGGCACAAGAUUUGAACA |
| | | CACGUUGAACCUAGAUGUAGGCAGCCUUCU |
| | | GGCAGUUUGUACACGGAAUGAGCACUCUUA |
| | | AUGCCACGCUAAGCGUUACAAUAUCAUUAU |
| | | AGUGCCAGGCAGCUGACGGAAGAAACGGUA |
| | | CCGUGGUGUAUCUUGAUCAGGCUUUCUUGA |
| | | UGCAGCCUGUGGGGGAACUUCAGUAGGCAG |
| | | GACGCACAGCUGUCACCAUAAACAGGUUGU |
| | | AGGGAGAGUAUCGGUCGACGCAAAUCUUGA |
| | | CCACGUCGCGCGUUGAAAGCCUUUCCUGGC |
| | | ACUCCAGCCUGUAGGCAGCACUCCAGAAAU |
| | | AUCUCAGAUCAAAAAGGGUGUAGAACGUGC |
| | | AUAGGUGCAACACACCUGUUGUGUUUUGUA |
| | | CGAGCUUGUGGCGUGAUCAAGUGGCGGUCA |
| | | GGAUUCUAGAGCGGCCGCUUCGAGCCGGUU |
| | | GAAUCGCUGAUCUCACGCCGUGGUGAGCUC |
| | | GCUUUCUUGCUGUCCAAUUUCUAUUAAAGG |
| | | UUCCUUUGUUCCCUAAGUCCAACUACUAAA |
| | | CUGGGGGAUAUUAUGAAGGGCCUUGAGCAU |
| | | CUGGAUUCUGCCUAAUAAAAAACAUUUAUU |
| | | UUCAUUGCAAAGUUCCGCGUACGUACGGCG |
| | | UC |
| FluM1 Epitope | SEQ ID NO: 73 | SEQ ID NO: 74 |
| | ACAUUUGCUUCUGACACAACUGUGUUCACUAGC | GAUCGACAAGACUUAACCACGAUUCCUGAU |
| | AACCUCAAACAGACACCACAUUUGCUUCUGACA | GCAUUGACUUACCAUCGACUCAACUGACAA |
| | CAACUGUGUUCACUAGCAACCUCAAACAGACAC | GGGACCACGCAGAGGUGAAUGAGUCAGGAC |
| | CGCCGCCACCAUGGGCAUCCUGGGCUUCGUGUU | UUUGUAGUCGGAGUCGGAAAAAACACCAGU |
| | CACCCUGUAGAGCUCGCUUUCUUGCUGUCCAAU | CACAAUGUAUCGUACGCUUGCUACUAGGAG |
| | UUCUAUUAAAGGUUCCUUUGUUCCCUAAGUCCA | CUCGUCAUGACGUUGAGAGCCUGUUAACUA |
| | ACUACUAAACUGGGGGAUAUUAUGAAGGGCCUU | GACACGUUCCUAAGGGUUAGCCACACAUUA |
| | GAGCAUCUGGAUUCUGCCUAAUAAAAAACAUUU | AUAUCGGGCCUGACACAGGACACGAAAAAA |
| | AUUUUCAUGCAGCAAGCUCGCUUUCUUGCUGUC | GAAGGUGCUGUUAGUUGGACAGGUACUAUC |
| | CAAUUUCUAUUAAAGGUUCCUUUGUUCCCUAAG | AUCUCAAGUCGAUAGUCCAAGUAGGUUUGA |
| | UCCAACUACUAAACUGGGGGAUAUUAUGAAGGG | ACCAUGCAUAGCUUGUAUCAGGUCAUCGCC |
| | CCUUGAGCAUCUGGAUUCUGCCUAAUAAAAAAC | UCAAACGUUAGGUGUCACAUUGUGGAAUCG |
| | AUUUAUUUUCAUGCAGCAAAUAAAAGCUCGCUU | CAAAAAACAUACCGACUUCCAUUAUGGGAC |
| | UCUUGCUGUCCAAUUUCUAUUAAAGGUUCCUUU | ACGUCGCUUAUUCUUGGUAAGUAGAAGUUG |
| | GUUCCCUAAGUCCAACUACUAAACUGGGGGAUA | CCAUCGUAGUCGCACGACCUACUUAUGACG |
| | UUAUGAAGGGCCUUGAGCAUCUGGAUUCUGCCU | AACUUCGGUUAAGUGGCUGACGUACUAACA |
| | AAUAAAAAACAUUUAUUUUCAU (poly-A150) | GUGCGUGCAAAAAAGACCUACGAAGCCAGA |
| | | GUUCGUUCCAGUGUGAAAGUGCACAUCACG |
| | | AGUUGUGCCAAUGCACGUUGCAUCGAGAGU |
| | | UAAUCCCGUCUUAAGUAGCAAGGCACCUGA |
| | | AUGGAAGUUGAUUCGUCUAGAAAUAGACGA |
| | | AUCAUGCUGAUCUCAGGUGCUCACUUGAUU |
| | | AAGACGGCUGUUUAUCUCGAUGCUUCCCCC |
| | | UUGGCACAAAUGCAUCAGUGCACUUAUGAU |
| | | AGUGAACGAACUCUGGCUUCGUAGGUCAAA |
| | | AAAGCACGCAAGCAUGUAACGUCAGCCUAA |
| | | CGCUUGAAGUUCGCAGGUGUGAGGUCGUGC |
| | | UUUGUGUGGCAACUGUCAUGACCCAAGAAU |
| | | AAGCGACGUGUCCCAUAGAUCAGCACGGUA |
| | | UGAAAAAAGCGAUUCGUGAGGUAGACACCU |
| | | AGAUACUCUGGCGAUGACUCGGCCAGCUAU |
| | | GCGAGUCGAUAACCUACUUGGACUAUCGAC |
| | | UUGAGUCACACUGACCUGUCCAUACAUGCU |
| | | CACCUUCAAAAAACGUGUCCGCACUAUAGC |
| | | CCGAUAUCUCGUACAGGCUAACCUUCUUCG |
| | | UGUCUAGUUAACAGGCUCUCAACUCUACUU |
| | | AGAGCUCCUAUCAAGUGACGUACGAUUACC |
| | | UCACACUGGUGAAAAAACCGACUCAAGAUU |
| | | UGAAGUCCUGGGCGACCUCUGCGUGGUCCC |
| | | UUGUCAGUUAUGGUUCAGGUAAGUCACUCG |
| | | UGAUGGAAUCGUAAGCGUUACUUGUCGAUU |
| | | AUAGUGCGCUGCUAAAAAAUGUUCUUGAUC |
| | | AGGCGUCCGUGCUCGGCCCCUUAGGAAUCC |
| | | CCCGGACUACGAUUGGCUCAUAAACAGGGC |
| | | CUAGAGUAUCUUGGCCAAAUCUUGCGGCAA |
| | | AAAACUGUUGUUUUUUUUACUCAUUCUCCG |
| | | UGCCUGAUACACUUCUAACGUGCAGUUUGU |
| | | CACACCUGUGGGCUGUACGAGCUUUUGUGA |
| | | UCAAGCGCGGCG |
| FluHA Epitope | SEQ ID NO: 75 | SEQ ID NO: 76 |
| | ACAUUUGCUUCUGACACAACUGUGUUCACUAGC | CUUAACCACGAUUCCUGAUGCAUUGACUUA |
| | AACCUCAAACAGACACCACAUUUGCUUCUGACA | CCAUCGACUCAACUGACAAGGGACCACGCA |
| | CAACUGUGUUCACUAGCAACCUCAAACAGACAC | GAGGUGAAUGAGUCAGGACUUUGUAGUCGG |
| | CGCCGCCACCAUGCCCAAGUACGUGAAGCAGAA | AGUCGGAAAAAACACCAGUCACAAUGUAUC |
| | CACCCUGAAGCUGGCCACCUAGAGCUCGCUUUC | GUACGCUUGCUACUAGGAGCUCGUCAUGAC |
| | UUGCUGUCCAAUUUCUAUUAAAGGUUCCUUUGU | GUUGAGAGCCUGUUAACUAGACACGUUCCU |
| | UCCCUAAGUCCAACUACUAAACUGGGGGAUAUU | AAGGGUUAGCCACACAUUAAUAUCGGGCCU |
| | AUGAAGGGCCUUGAGCAUCUGGAUUCUGCCUAA | GACACAGGACACGAAAAAAGAAGGUGCUGU |
| | UAAAAAACAUUUAUUUUCAUGCAGCAAGCUCGC | UAGUUGGACAGGUACUAUCAUCUCAAGUCG |
| | UUUCUUGCUGUCCAAUUUCUAUUAAAGGUUCCU | AUAGUCCAAGUAGGUUUGAACCAUGCAUAG |
| | UUGUUCCCUAAGUCCAACUACUAAACUGGGGGA | CUUGUAUCAGGUCAUCGCCUCAAACGUUAG |
| | UAUUAUGAAGGGCCUUGAGCAUCUGGAUUCUGC | GUGUCACAUUGUGGAAUCGCAAAAAACAUA |
| | CUAAUAAAAAACAUUUAUUUUCAUGCAGCAAAU | CCGACUUCCAUUAUGGGACACGUCGCUUAU |
| | AAAAGCUCGCUUUCUUGCUGUCCAAUUUCUAUU | UCUUGGUAAGUAGAAGUUGCCAUCGUAGUC |
| | AAAGGUUCCUUUGUUCCCUAAGUCCAACUACUA | GCACGACCUACUUAUGACGAACUUCGGUUA |
| | AACUGGGGGAUAUUAUGAAGGGCCUUGAGCAUC | AGUGGCUGACGUACUAACAGUGCGUGCAAA |
| | UGGAUUCUGCCUAAUAAAAAACAUUUAUUUUCA | AAAGACCUACGAAGCCAGAGUUCGUUCCAG |
| | U (poly-A150) | UGUGAAAGUGCACAUCACGAGUUGUGCCAA |
| | | UGCACGUUGCAUCGAGAGUUAAUCCCGUCU |
| | | UAAGUAGCAAGGCACCUGAAUGGAAGUUGA |
| | | UUCGUCUAGAAAUAGACGAAUCAUGCUGAU |
| | | CUCAGGUGCUCACUUGAUUAAGACGGCUGU |
| | | UUAUCUCGAUGCUUGUGUUUGGCACAAAUG |
| | | CAUCAGUGCACUUAUGAUAGUGAACGAACU |
| | | CUGGCUUCGUAGGUCAAAAAAGCACGCAAG |
| | | CAUGUAACGUCAGCCUAACGCUUGAAGUUC |
| | | GCAGGUGUGAGGUCGUGCGCUUGGUGGCAA |
| | | CUGUCAUGACCCAAGAAUAAGCGACGUGUC |
| | | CCAUAGAUCAGCACGGUAUGAAAAAAGCGA |
| | | UUCGUGAGGUAGACACCUAGAUACUCUGGC |
| | | GAUGACCUUUAGCUAUGCGAGUCGAUAACC |
| | | UACUUGGACUAUCGACUUGAGUCACACUGA |
| | | CCUGUCCAUACAUGCUCACCUUCAAAAAAC |
| | | GUGUCCGCACUAUAGCCCGAUAUCUCGUAC |
| | | AGGCUAACCCCCCGUUCGUGUCUAGUUAAC |
| | | AGGCUCUCAACUCUACUUAGAGCUCCUAUC |
| | | AAGUGACGUACGAUUACCUCACACUGGUGA |
| | | AAAAACCGACUCAAGAUUUGAAGUCCUGGU |
| | | GCUCACCUCUGCGUGGUCCCUUGUCAGUUA |
| | | UGGUUCAGGUAAGUCACUCGUGAUGGAAUC |
| | | GUAAGCGUUAUUUUUGCUUAUAGUGCUUGA |
| | | AAAAAUUGGCCUUGAUCAGUGCUCGGCCCU |
| | | UCUUUCCUUAGGAAUGUGUUGGACUACGAG |
| | | CCAUAAACAGUGUAGAGUAUCGGCCAAAUC |
| | | UUUGCUGAAAAAACUGUUUUUUUUGGCGUC |
| | | CACUCAUUCGGCCCUUCUGAUACACCCCCG |
| | | UAACGUGCAUGGCUUGCACACCUGGGCCUU |
| | | UGUACGAGUUGGCGUGAUCAAGCGGCUCUG |
| | | GUG |

*All mRNA sequences have a poly-A₁₅₀ tail (which is not provided in the SEQUENCE LISTING).

What is claimed:

1. A nanoparticle composition comprising a folded monodispersed nucleic acid nanostructure.

2. The composition of claim 1, wherein the nucleic acid nanostructure comprises a messenger RNA (mRNA), a single-stranded DNA (ssDNA), or a combination thereof.

3. The composition of claim 1, wherein the nucleic acid nanostructure comprises one or more nucleic acid sequences having at least 90-95% identity to SEQ ID NO: 1, 3, 5, 7, 9, 11, 13, or 15-76.

4. The composition of claim 1, wherein the nucleic acid nanostructure comprises one or more nucleic acid sequences of SEQ ID NO: 1, 3, 5, 7, 9, 11, 13, or 15-76

5. The composition of claim 1, further comprising one or more DNA or RNA staple strands.

6. The composition of claim 5, wherein the staple strands are short DNA staple strands.

7. The composition of claim 1, wherein the nucleic acid nanostructure comprises a single-stranded mRNA that serves as a scaffold strand, and one or more short DNA or RNA staple strands that promote folding and stabilization of the mRNA into a compact monodispersed nanostructure.

8. The composition of claim 1, wherein the folded monodispersed nucleic acid nanostructure comprises a 6-helix bundle (6HB), a 12-helix bundle, a DNA octahedron, or a combination thereof.

9. The composition of claim 1, wherein the folded monodispersed nucleic acid nanostructure is coated with one or more peptides selected from a poly-lysine peptide, a poly-lysine peptide having a PEG chain, a poly-aurein peptide, a poly-aurein peptide having a PEG chain, a specific cell-targeting peptide, an endosomal escape peptide, and combinations thereof.

10. The composition of claim 9, wherein the endosomal escape peptide has an amino acid sequence having at least 90-95% identity to SEQ ID NO: 77-81.

11. The composition of claim 9, wherein the endosomal escape peptide has an amino acid sequence of SEQ ID NO: 77-81.

12. The composition of claim 9, wherein the endosomal escape peptide comprises a lysine10 (K10) peptide flanked by two copies of an aurein 1.2 peptide (SEQ ID NO: 77).

13. The composition of claim 9, wherein the poly-lysine peptide comprises a lysine 10 (K10) peptide (SEQ ID NO: 78).

14. The composition of claim 1, wherein the nucleic acid nanostructure has intramolecular folding capabilities without the need for helper nucleic acid strands.

15. A method of improving cellular uptake of a nucleic acid nanostructure, the method comprising delivering to a cell a nanoparticle composition comprising a folded monodispersed nucleic acid nanostructure comprising a messenger RNA (mRNA), a single-stranded DNA (ssDNA), or a combination thereof.

16. The method of claim 15, wherein the folded monodispersed nucleic acid nanostructure is coated with one or more peptides selected from a poly-lysine peptide, a poly-lysine peptide having a PEG chain, a poly-aurein peptide, a poly-aurein peptide having a PEG chain, a specific cell-targeting peptide, an endosomal escape peptide, and combinations thereof.

17. The method of claim 15, wherein the nucleic acid nanostructure further comprises one or more DNA or RNA staple strands to promote folding and stabilization of the nucleic acid nanostructure.

18. The method of claim 15, wherein the nucleic acid nanostructure comprises a single-stranded mRNA that serves as a scaffold strand, and one or more short DNA staple strands that promote folding and stabilization of the mRNA into a compact monodispersed nanostructure.

19. The method of claim 15, where the nanostructure comprises mRNA with a designed single-stranded RNA to fold it into a compact nanostructure comprised of double-stranded regions, loops and cross-overs.

20. The method of claim 15, where the nanostructure comprises a designed single-stranded RNA wherein one half comprises the designed mRNA and second half comprises the designed regions to drive folding of the entire single-stranded RNA into a compact nanostructure comprised of double-stranded regions, loops and cross-overs.


SEQUENCE LISTING (81 sequences)

SEQ ID NO: 1 - RNA, 720 nt, synthetic construct
  misc_feature 1..720: note = Synthetic
  source 1..720: mol_type = other RNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 2 - protein, 239 aa, synthetic construct
  REGION 1..239: note = Synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 3 - RNA, 633 nt, synthetic construct
  misc_feature 1..633: note = Synthetic
  source 1..633: mol_type = other RNA
atgtggctcgtttccttagcaatagtgacagcctgcgccggggcaaccaacctttgcccctttggggaggtattcaatgctactagattcgctagcgtgtacgcttggaatcgtaagagaatttccaattgtgttgcagactactcagtgctttataactctgcaagcttctctacatttaaatgttatggcgtgtcacctacaaagctgaatgatttatgcttcacgaacgtctacgcggatagcttcgttatcaggggggacgaagtgaggcagattgcccccggtcagaccggtaagatcgcagattacaactacaagctgccagacgatttcactggctgcgtgatagcttggaatagcaacaacttagacagcaaggtgggtggcaactataactatctgtacagactctttcggaagtctaatcttaagcccttcgaaagagacataagcacggagatctatcaagcagggagtacaccgtgtaatggtgtcgaaggcttcaattgttatttccctctgcaaagttacggttttcaacccaccaatggagtgggataccagccatacagggtagttgtgttgtcatttgagctgttacacgctcctgccactgtgtgcgggccttaa

SEQ ID NO: 4 - protein, 210 aa, synthetic construct
  REGION 1..210: note = Synthetic
MWLVSLAIVTACAGATNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 5 - RNA, 3912 nt, synthetic construct
  misc_feature 1..3912: note = Synthetic
  source 1..3912: mol_type = other RNA
atgtggctggtgagcctcgccattgtgacggcctgcgccggggccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggcagcgccagcagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacccccctgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcaggggtctggatacattccagaggctcccagggacggccaggcctatgttaggaaggatggtgagtgggtcctgctgagtaccttcctcggcagatccctggaggtgctgtttcagggcccggggcaccaccaccatcatcaccatcacagtgcctggagtcatcctcaattcgagaagggaggcggaagcggagggggaggctccggaggaagcgcttggtcccaccctcaattcgaaaaataa

SEQ ID NO: 6 - protein, 1303 aa, synthetic construct
  REGION 1..1303: note = Synthetic
MWLVSLAIVTACAGAMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 7 - RNA, 448 nt, synthetic construct
  misc_feature 1..448: note = Synthetic
  source 1..448: mol_type = other RNA
acatttgcttctgacacaactgtgttcactagcaacctcaaacagacaccgccgccaccatgcggcacctggacaagaagcagagattccacaacatcagaggcagatggaccggccgttgcatgagctgctgcagaagcagcagaaccagaagagagacacagctgtagagctcgctttcttgctgtccaatttctattaaaggttcctttgttccctaagtccaactactaaactgggggatattatgaagggccttgagcatctggattctgcctaataaaaaacatttattttcatgcagcaagctcgctttcttgctgtccaatttctattaaaggttcctttgttccctaagtccaactactaaactgggggatattatgaagggccttgagcatctggattctgcctaataaaaaacatttattttcatgcagcaaataaa

SEQ ID NO: 8 - protein, 35 aa, synthetic construct
  REGION 1..35: note = Synthetic
RHLDKKQRFHNIRGRWTGRCMSCCRSSRTRRETQL

SEQ ID NO: 9 - RNA, 1206 nt, synthetic construct
  misc_feature 1..291: note = Synthetic
  source 1..1206: mol_type = other RNA
acatttgcttctgacacaactgtgttcactagcaacctcaaacagacaccatggggatcctgggcttcgtgtttaccctcgctgcctacatcctgaccgccttcaactccagccacaaggctgcctacaacacaacccccatcgtgcacctgaaggctgcctacaccctcaagtgcctgagatacaggttcaaagctgcctacaggctggagtgcgccatctactataaggctgcctacgtggttgagggccaggtcgactactatgctgcctacaagagcgccatcgtgacactgacctacgctgcctacctgaccgcccccacaggctgcatcaagaaagctgcctacacatacaccagcctcattatcctggtgttggctgcctacaccattcatgatattattctggaatgcgtggctgcctacctgctcattaggtgtatcaactgccagaaggctgcctacatcattctggagtgtgtgtactgcaaggctgcctactgcgtgtactgtaagcagcaactcctgagggctgcctacacaaccctggagcagcaatacaacaaggctgcctacgccgtgtgcgacaaatgtctgaagttctacgctgcctacgccgagcccgacagggctcactacaacatcgtgaccttctgttgcaaggctgcctacggcaccctgggaatcgtgtgtcccatttgcagccagaaggctgcctacttcgtgtacatccccctgtttttgattcacacccatgccaggttcctcataggacccggccctgggatcaggccccttctgctgtccgtgagcacctacacatctctcataattggacccggccctggggatagcacactcaggctgtgcgtgcagtccacccacgtcgacatcagaactcttttggaggacttactgatgggacccggccctgggtattgctacgaacagctgaacgactccagcgaggaggaggataccgacgaaatcgattaatctagagcggccgcttcgagccggttgaatcgctgatctcacgccgtggtgagctcgctttcttgctgtccaatttctattaaaggttcctttgttccctaagtccaactactaaactgggggatattatgaagggccttgagcatctggattctgcctaataaaaaacatttattttcattgcaaagttccgcgtacgtacggcgtc

SEQ ID NO: 10 - protein, 226 aa, synthetic construct
  REGION 1..226: note = Synthetic
MPKYVKQNTLKLATHGDTPTLHEYMLDLQPETTTGNPCHTTKLLHRDSVDSAPILTAFNSSHKGASVTVVEGQVDYYGLYYVHEGIRTYRHLDKKQRFHNIRGRWTGRCMSCCRSSRTRRETQLQYSNEKWTLQDVSLEVYLTAPTGCIKKNTTPIVHLKGDANTLKCLRYRFKKHCTLYTAVSSTWFAFRDLCIVYRDGNPYAVCDKCLKFYSKISGILGFVFTL

SEQ ID NO: 11 - RNA, 291 nt, synthetic construct
  misc_feature 1..291: note = Synthetic
  source 1..291: mol_type = other RNA
acatttgcttctgacacaactgtgttcactagcaacctcaaacagacaccatggggatcctgggcttcgtgtttaccctctaatctagagcggccgcttcgagccggttgaatcgctgatctcacgccgtggtgagctcgctttcttgctgtccaatttctattaaaggttcctttgttccctaagtccaactactaaactgggggatattatgaagggccttgagcatctggattctgcctaataaaaaacatttattttcattgcaaagttccgcgtacgtacggcgtc

SEQ ID NO: 12 - protein, 8 aa, synthetic construct
  REGION 1..8: note = Synthetic
GILGFVFT

SEQ ID NO: 13 - RNA, 382 nt, synthetic construct
  misc_feature 1..382: note = Synthetic
  source 1..382: mol_type = other RNA
acatttgcttctgacacaactgtgttcactagcaacctcaaacagacaccgccgccaccatgcccaagtacgtgaagcagaacaccctgaagctggccacctagagctcgctttcttgctgtccaatttctattaaaggttcctttgttccctaagtccaactactaaactgggggatattatgaagggccttgagcatctggattctgcctaataaaaaacatttattttcatgcagcaagctcgctttcttgctgtccaatttctattaaaggttcctttgttccctaagtccaactactaaactgggggatattatgaagggccttgagcatctggattctgcctaataaaaaacatttattttcatgcagcaaataaa

SEQ ID NO: 14 - protein, 13 aa, synthetic construct
  REGION 1..13: note = Synthetic
PKYVKQNTLKLAT

SEQ ID NO: 15 - RNA, 254 nt, synthetic construct
  misc_feature 1..254: note = Synthetic
  source 1..254: mol_type = other RNA
gggacatttgcttctgacacaactgtgttcactagcaacctcaaacagacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctac

SEQ ID NO: 16 - DNA, 38 bp, synthetic construct
  misc_feature 1..38: note = Synthetic
  source 1..38: mol_type = other DNA
gggtgggcgaacttcattgtgtcagaagcaaatgtccc

SEQ ID NO: 17 - DNA, 45 bp, synthetic construct
  misc_feature 1..45: note = Synthetic
  source 1..45: mol_type = other DNA
gcaagaaatagtagttcgccgtacgtacgcgaacacaggggtcag

SEQ ID NO: 18 - DNA, 42 bp, synthetic construct
  misc_feature 1..42: note = Synthetic
  source 1..42: mol_type = other DNA
gctagtggaactttaatatcccccagttgcgagctgtcacga

SEQ ID NO: 19 - DNA, 45 bp, synthetic construct
  misc_feature 1..45: note = Synthetic
  source 1..45: mol_type = other DNA
ctcaaggtcagcgactgcacgccgtaggttaggtggcttgaggtt

SEQ ID NO: 20 - DNA, 42 bp, synthetic construct
  misc_feature 1..42: note = Synthetic
  source 1..42: mol_type = other DNA
aataaattgtctgtatcgccctcgcccttgaagcattcaacc

SEQ ID NO: 21 - DNA, 43 bp, synthetic construct
  misc_feature 1..43: note = Synthetic
  source 1..43: mol_type = other DNA
accatgggttttttattaggcatggtcggtcctcgcacgctga

SEQ ID NO: 22 - DNA, 46 bp, synthetic construct
  misc_feature 1..46: note = Synthetic
  source 1..46: mol_type = other DNA
ggctcgaagcggccgggtcggggtagcggccgccggacccttgctc

SEQ ID NO: 23 - DNA, 43 bp, synthetic construct
  misc_feature 1..43: note = Synthetic
  source 1..43: mol_type = other DNA
gaacagccgagctgggggtgttctgctggtaggaatccagatg

SEQ ID NO: 24 - DNA, 45 bp, synthetic construct
  misc_feature 1..45: note = Synthetic
  source 1..45: mol_type = other DNA
gggccgttacagctaagaagtcgtgctgcgccgtttaaccccggt

SEQ ID NO: 25 - DNA, 43 bp, synthetic construct
  misc_feature 1..43: note = Synthetic
  source 1..43: mol_type = other DNA
cgtcctcgggcacccgtcgccgtccagctcgatcagctcgatg

SEQ ID NO: 26 - DNA, 43 bp, synthetic construct
  misc_feature 1..43: note = Synthetic
  source 1..43: mol_type = other DNA
cttgaagtgaagtcgatgccctccaggatgatgttgcagcacg

SEQ ID NO: 27 - DNA, 42 bp, synthetic construct
  misc_feature 1..42: note = Synthetic
  source 1..42: mol_type = other DNA
tcctcctttcaccttcaggtagtggttgtgatcccgccttcg

SEQ ID NO: 28 - DNA, 45 bp, synthetic construct
  misc_feature 1..45: note = Synthetic
  source 1..45: mol_type = other DNA
gggcggactccagcagatggtgcgctcctccagggtgtgttgccg

SEQ ID NO: 29 - DNA, 57 bp, synthetic construct
  misc_feature 1..57: note = Synthetic
  source 1..57: mol_type = other DNA
ttcttctcccaggatcgccctcgaacttttgaagaaggaccatgtgatcgcgcttct

SEQ ID NO: 30 - DNA, 52 bp, synthetic construct
  misc_feature 1..52: note = Synthetic
  source 1..52: mol_type = other DNA
tagttgccgtcgtcccacctcggtactccagcttgtgcgcttgtcttgctca

SEQ ID NO: 31 - DNA, 22 bp, synthetic construct
  misc_feature 1..22: note = Synthetic
  source 1..22: mol_type = other DNA
tgttgtagttgcgcgggtcttg

SEQ ID NO: 32 - DNA, 32 bp, synthetic construct
  misc_feature 1..32: note = Synthetic
  source 1..32: mol_type = other DNA
aaaaaaaagaggacttagggaacaaaggaacc

SEQ ID NO: 33 - DNA, 45 bp, synthetic construct
  misc_feature 1..45: note = Synthetic
  source 1..45: mol_type = other DNA
cttgccgcagggtgcaccacggcgtgagacccttcatgcaatgaa

SEQ ID NO: 34 - DNA, 45 bp, synthetic construct
  misc_feature 1..45: note = Synthetic
  source 1..45: mol_type = other DNA
acttgtgttcatgtctctagaattacttgcgccgatgcacgctgc

SEQ ID NO: 35 - DNA, 46 bp, synthetic construct
  misc_feature 1..46: note = Synthetic
  source 1..46: mol_type = other DNA
ggcatggcggacttgcgtccatgccgagagtcgggcagtggcggat

SEQ ID NO: 36 - DNA, 45 bp, synthetic construct
  misc_feature 1..45: note = Synthetic
  source 1..45: mol_type = other DNA
cggttcaggacgtaggcggcggtcacgaactgggtgctgatgccg

SEQ ID NO: 37 - DNA, 34 bp, synthetic construct
  misc_feature 1..34: note = Synthetic
  source 1..34: mol_type = other DNA
cgttggggtctggccatgatatagacgttgtggc

SEQ ID NO: 38 - DNA, 22 bp, synthetic construct
  misc_feature 1..22: note = Synthetic
  source 1..22: mol_type = other DNA
tttaatagaaacgggcagcttg

SEQ ID NO: 39 - DNA, 28 bp, synthetic construct
  misc_feature 1..28: note = Synthetic
  source 1..28: mol_type = other DNA
ccggtggtgcagatcagggcattggaca

SEQ ID NO: 40 - DNA, 1008 bp, synthetic construct
  misc_feature 1..1008: note = Synthetic
  source 1..1008: mol_type = other DNA
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcct

SEQ ID NO: 41 - DNA, 52 bp, synthetic construct
  misc_feature 1..52: note = Synthetic
  source 1..52: mol_type = other DNA
caaatggtcaattttttaacctgtttaggtggcatcatttttattctactaa

SEQ ID NO: 42 - DNA, 42 bp, synthetic construct
  misc_feature 1..42: note = Synthetic
  source 1..42: mol_type = other DNA
ttttcatttctcatagctgggggcgcgagctgaaaagctata

SEQ ID NO: 43 - DNA, 51 bp, synthetic construct
  misc_feature 1..51: note = Synthetic
  source 1..51: mol_type = other DNA
ttaaatagcatttttactaaagtacgattcccaatttttttctgcgaacga

SEQ ID NO: 44 - DNA, 42 bp, synthetic construct
  misc_feature 1..42: note = Synthetic
  source 1..42: mol_type = other DNA
catataacagttggtgtctggaagtttctacctgagagattc

SEQ ID NO: 45 - DNA, 52 bp, synthetic construct
  misc_feature 1..52: note = Synthetic
  source 1..52: mol_type = other DNA
tagagcttaatttttttgctgaatattgaccttcatctttttaagagtaatc

SEQ ID NO: 46 - DNA, 52 bp, synthetic construct
  misc_feature 1..52: note = Synthetic
  source 1..52: mol_type = other DNA
ttaattgctcctttttttttgataaggcatcaaaaagtttttattaagagga

SEQ ID NO: 47 - DNA, 52 bp, synthetic construct
  misc_feature 1..52: note = Synthetic
  source 1..52: mol_type = other DNA
acttcaaatattttttcgcgttttaaaactccaacagtttttgtcaggatta

SEQ ID NO: 48 - DNA, 42 bp, synthetic construct
  misc_feature 1..42: note = Synthetic
  source 1..42: mol_type = other DNA
aagagcccatccccctcaaatgctcataaatattcattgaga

SEQ ID NO: 49 - DNA, 52 bp, synthetic construct
  misc_feature 1..52: note = Synthetic
  source 1..52: mol_type = other DNA
taacgccaaaatttttggaattacgaaggtctttacctttttctgactatta

SEQ ID NO: 50 - DNA, 52 bp, synthetic construct
  misc_feature 1..52: note = Synthetic
  source 1..52: mol_type = other DNA
cgtccaatacttttttgcggaatcgtttaaacagttctttttagaaaacgag

SEQ ID NO: 51 - DNA, 52 bp, synthetic construct
  misc_feature 1..52: note = Synthetic
  source 1..52: mol_type = other DNA
tcataaccctctttttgtttaccagatgcaaaagaagtttttttttgccaga

SEQ ID NO: 52 - DNA, 42 bp, synthetic construct
  misc_feature 1..42: note = Synthetic
  source 1..42: mol_type = other DNA
atcaagataatagcgagaggctttcgacgataaaaaccaatc

SEQ ID NO: 53 - DNA, 52 bp, synthetic construct
  misc_feature 1..52: note = Synthetic
  source 1..52: mol_type = other DNA
agttgagattttttttaggaataccattacccaaatctttttaacgtaacaa

SEQ ID NO: 54 - DNA, 52 bp, synthetic construct
  misc_feature 1..52: note = Synthetic
  source 1..52: mol_type = other DNA
tcagtgaataatttttggcttgccctaacaacattatttttttacaggtaga

SEQ ID NO: 55 - DNA, 52 bp, synthetic construct
  misc_feature 1..52: note = Synthetic
  source 1..52: mol_type = other DNA
ttttaagaacttttttggctcattatacgttgggaagtttttaaaaatctac

SEQ ID NO: 56 - DNA, 52 bp, synthetic construct
  misc_feature 1..52: note = Synthetic
  source 1..52: mol_type = other DNA
tagtaaattggtttttgcttgagatgaactttaatcatttttttgtgaatta

SEQ ID NO: 57 - DNA, 64 bp, synthetic construct
  misc_feature 1..64: note = Synthetic
  source 1..64: mol_type = other DNA
ttgcggatggcttagtcagaagcaaagcggattaggtcatttgttgttttacttgtacagctcg

SEQ ID NO: 58 - DNA, 64 bp, synthetic construct
  misc_feature 1..64: note = Synthetic
  source 1..64: mol_type = other DNA
taaatcaaaaatcggcatagtaagagcaacactaaatgaccagttgttcagggtgtcgccctcg

SEQ ID NO: 59 - DNA, 64 bp, synthetic construct
  misc_feature 1..64: note = Synthetic
  source 1..64: mol_type = other DNA
taatgcagatacattgacaagaaccggatattcacattcaacgttgttcacgctgaacttgtgg

SEQ ID NO: 60 - DNA, 64 bp, synthetic construct
  misc_feature 1..64: note = Synthetic
  source 1..64: mol_type = other DNA
gcattaggctggcaatgctgtagctcaacatgtttagtagtagttgttgttgtggctgttgtac

SEQ ID NO: 61 - DNA, 64 bp, synthetic construct
  misc_feature 1..64: note = Synthetic
  source 1..64: mol_type = other DNA
tagtaaaatcaggaccagtgtttagactggataggggggtaagttgttgggcatggcggacttg

SEQ ID NO: 62 - DNA, 64 bp, synthetic construct
  misc_feature 1..64: note = Synthetic
  source 1..64: mol_type = other DNA
caccagaacgaggttaataaaacgaactaacgggacgagaaagttgttggtgggccagggcacg

SEQ ID NO: 63 - DNA, 64 bp, synthetic construct
  misc_feature 1..64: note = Synthetic
  source 1..64: mol_type = other DNA
agtttgacatttcgtttacattagatacatttcggtagatttgttgttggcgagctgcacgctg

SEQ ID NO: 64 - DNA, 64 bp, synthetic construct
  misc_feature 1..64: note = Synthetic
  source 1..64: mol_type = other DNA
caaagcgtgcgaccttaaaccagaccggaagcattcgagcttgttgtttttgctcagggcggac

SEQ ID NO: 65 - RNA, 823 nt, synthetic construct
  misc_feature 1..823: note = Synthetic
  source 1..823: mol_type = other RNA
acatttgcttctgacacaactgtgttcactagcaacctcaaacagacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagggacatttgcttctgacacaactgtgttcactagcaacctcaaacagacacc

SEQ ID NO: 66 - RNA, 1109 nt, synthetic construct
  misc_feature 1..1109: note = Synthetic
  source 1..1109: mol_type = other RNA
taagtagaagttgccatcgtagtcgcacgacctacttatgacgaacttcggttaagtggctgacgtcctaacagtgcgtgcaaaaaagacctacgaagccagagttcgttccagtgtgaaagtgcacatcacgagttgtgccaatgcacgttgcatcgagagttaatcccgtcttaagtagcaaggcacctgaatggaagttgattcgtctagcaatagacgaatcatgctgatctcaggtgctcacttgattaagacggctgtttatctcgatgctcgccctcttggcacaatcgaacttgtgcacttcagcacgggaacgaactctggcttcgtaggtcaaaaaagcacgcaagcatgtaacgtcagcctaacgcttgaagttcgcaggtgtgaggtcgtgcatgtggtctggcaactctgcttgtttacttgtacagctcgtccatgccggatcagcacccggcgaaaaaaacgaactgtgaggtaaccatgtggatactcttctcgttggcttgccgtctcagggctcaccaggtgctcaggtagtggttgtcgggcagtcacactgggccgtcgatacatgctgtgtttcaaaaaacgtggtcgcactatagcacgctgcctcgtacaatgtagcgagaagtcgtgaagttcaccttgatgccgttctttctacttacggccatgatcaagtgattgtggcttacctcactgtactgaaaaaagtgccccaagatttggccgtcctatgaactttcgatgcccttcagctcgatgcggtggactggggtgtcgcccctcgtgatcacctcggaagcgttattgtagttgtatagtgccttgaagaaaaaagtgcgctcttgatcatagccttcgggcatggcggacttgagcggatcttgctgcttcgactacgaggggtagcataaacagcactgcacgagagtatccagggtggcaaatcttgtgggccaaaaaacgggcagcttgccggtggtgcagccttgaagcagggtcagggtctttgaggtggcaaacgtgcagccctcgcccacacctgtgaacttgtgtacgagtacgtcgcctgatcaagcgaccaggatggg

SEQ ID NO: 67 - RNA, 895 nt, synthetic construct
  misc_feature 1..895: note = Synthetic
  source 1..895: mol_type = other RNA
gggacatttgcttctgacacaactgtgttcactagcaacctcaaacagacaccatgtggctcgtttccttagcaatagtgacagcctgcgccggggcaaccaacctttgcccctttggggaggtattcaatgctactagattcgctagcgtgtacgcttggaatcgtaagagaatttccaattgtgttgcagactactcagtgctttataactctgcaagcttctctacatttaaatgttatggcgtgtcacctacaaagctgaatgatttatgcttcacgaacgtctacgcggatagcttcgttatcaggggggacgaagtgaggcagattgcccccggtcagaccggtaagatcgcagattacaactacaagctgccagacgatttcactggctgcgtgatagcttggaatagcaacaacttagacagcaaggtgggtggcaactataactatctgtacagactctttcggaagtctaatcttaagcccttcgaaagagacataagcacggagatctatcaagcagggagtacaccgtgtaatggtgtcgaaggcttcaattgttatttccctctgcaaagttacggttttcaacccaccaatggagtgggataccagccatacagggtagttgtgttgtcatttgagctgttacacgctcctgccactgtgtgcgggccttaattctagagcggccgcttcgagccggttgaatcgctgatctcacgccgtggtgagctcgctttcttgctgtccaatttctattaaaggttcctttgttccctaagtccaactactaaactgggggatattatgaagggccttgagcatctggattctgcctaataaaaaacatttattttcattgcaaagttccgcgtacgtacggcgtc

SEQ ID NO: 68 - RNA, 1081 nt, synthetic construct
  misc_feature 1..1081: note = Synthetic
  source 1..1081: mol_type = other RNA
ggtcatcgcctcaaacgttaggtgtcacattgtggaatcgcaaaaaacataccgacttccattatgggacacgtcgcttattcttggtaagtagaagttgccatcgtagtcgcacgacctacttatgacgaacttcggttaagtggctgacgtgctaacagtgcgtgcaaaaaagacctacgaagccagagttcgttccagtgtgaaagtgcacatcacgagttgtgccaatgcacgttgcatcgagagttaatcccgtcttaagtagcaaggcacctgaatggaagttgattcgtctagaaatagacgaatcatgctgatctcaggtgctcacttgattaagacggctgtttatctcgatgcttccgcttggcacaacgcgccgtgcacttccctgaacgaactctggcttcgtaggtcaaaaaagcacgcaagcatgtaacgtcagcctaacgcttgaagttcgcaggtgtgaggtcgtgctgcgtttggcaacttgcttgtccaagaataagcgacgtgtcccatagatcagcacggtatgaaaaaagcgattcgtgaggtagacacctagatactctggcgatgacggttcccgccctgctttcggcgtgtcgctctgctcacactgccctgttatacatgctcccttcaaaaaacgggttggcactatactttgcctcgtacattacttcggcgcccttccggtgtctccctctacttagtctccgtcaagtgatctctttctacctcactgtgaaaaaaccggaagatttgcgtgttgtctgccccccccttgctgtctgttgtgtggcgcgcttcctcgtgatgtgtaagcgttagcttgtgtatagtgctgcgtcaaaaaagtctgccttgatcatctgcctccttcgtcccccctgggccttcttccgcggactacgacgtggcataaacagttcgctttagagtatcccgcctcaaatctttgtgaaaaaactgcggtttgcctggttgttgccttcttggctcttcgaacgtgcagtccgctcacacctgtgtgctgtacgagctcccctgatcaagggttggttgcccc

SEQ ID NO: 69 - RNA, 628 nt, synthetic construct
  misc_feature 1..628: note = Synthetic
  source 1..628: mol_type = other RNA
acatttgcttctgacacaactgtgttcactagcaacctcaaacagacaccacatttgcttctgacacaactgtgttcactagcaacctcaaacagacaccgccgccaccatgcggcacctggacaagaagcagagattccacaacatcagaggcagatggaccggccgttgcatgagctgctgcagaagcagcagaaccagaagagagacacagctgtagagctcgctttcttgctgtccaatttctattaaaggttcctttgttccctaagtccaactactaaactgggggatattatgaagggccttgagcatctggattctgcctaataaaaaacatttattttcatgcagcaagctcgctttcttgctgtccaatttctattaaaggttcctttgttccctaagtccaactactaaactgggggatattatgaagggccttgagcatctggattctgcctaataaaaaacatttattttcatgcagcaaataaaagctcgctttcttgctgtccaatttctattaaaggttcctttgttccctaagtccaactactaaactgggggatattatgaagggccttgagcatctggattctgcctaataaaaaacatttattttcat

SEQ ID NO: 70 - RNA, 1354 nt, synthetic construct
  misc_feature 1..1354: note = Synthetic
  source 1..1354: mol_type = other RNA
aatgagtcaggactttgtagtcggagtcggaaaaaacaccagtcacaatgtatcgtacgcttgctactaggagctcgtcatgacgttgagagcctgttaactagacacgttcctaagggttagccacacattaatatcgggcctgacacaggacacgaaaaaagaaggtgctgttagttggacaggtactatcatctcaagtcgatagtccaagtaggtttgaaccatgcatagcttgtatcaggtcatcgcctcaaacgttaggtgtcacattgtggaatcgcaaaaaacataccgacttccattatgggacacgtcgcttattcttggtaagtagaagttgccatcgtagtcgcacgacctacttatgacgaacttcggttaagtggctgacgtactaacagtgcgtgcaaaaaagacctacgaagccagagttcgttccagtgtgaaagtgcacatcacgagttgtgccaatgcacgttgcatcgagagttaatcccgtcttaagtagcaaggcacctgaatggaagttgattcgtctagaaatagacgaatcatgctgatctcaggtgctcacttgattaagacggctgtttatctcgatgccgctgtttggcacaaccgtgcgtgcacttatgatagtgaacgaactctggcttcgtaggtcaaaaaagcacgcaagcatgtaacgtcagcctaacgcttgaagttcgcaggtgtgaggtcgtgcttgttggcaactgtcatgacccaagaataagcgacgtgtcccatagatcagcacggtatgaaaaaagcgattcgtgaggtagacacctagatactctggcgatgaccggagctatgctttttttaacctacttggactatcgacttgagtcacactgacctgtccatacatgctcaccttcaaaaaacgtgtccgcactatagcccgatatctcgtacaggctaaccggcttgccgtgtctagttaacaggctctcaactctacttagagctcctatcaagtgacgtacgattacctcacactggtgaaaaaaccgactcaagatttgaagtcctgttgtttttgctgctgttgtatggttcaggcgtctcgtgattcggccaagcgttattccccctatagtgcgttggcaaaaaacgcttgatcatgttggcgcggccttaggaatgctggactacgattttttcataaacagtccgtgcagagtatccttctcaaatcttgtttgaaaaaaccttgggcggccttactcatttttggcgctgatacacggctctaacgtgcagtctctcttcacacctggctgcttctgtacgagctctgctgatcaagcctctgcctctg

SEQ ID NO: 71 - RNA, 1306 nt, synthetic construct
  misc_feature 1..1306: note = Synthetic
  source 1..1306: mol_type = other RNA
acatttgcttctgacacaactgtgttcactagcaacctcaaacagacaccatgcgggtgaccgccccccggaccctgatcctgctgctgagcggcgccctggccctgaccgaaacctgggccggcagcggcggcagcggcggcggcagcggtggacccaagtacgtgaagcagaacaccctgaagctggccacccacggcgacacccctaccctgcacgagtacatgctggacctgcagcctgagacaaccaccggcaacccctgccacaccaccaagctgctgcacagagatagcgtggacagcgcccctatcctgaccgccttcaacagcagccacaagggcgccagcgtgaccgtggtggaaggccaggtggactactacggcctgtactacgtgcacgagggcatcagaacctaccggcacctggacaagaagcagagattccacaacatcagaggcagatggaccggccgttgcatgagctgctgcagaagcagcagaaccagaagagagacacagctgcaatacagcaacgagaagtggaccctgcaggacgtgtccctggaagtgtacctgaccgcccctaccggctgcatcaagaagaataccacccctatcgtgcacctgaaaggcgacgccaacaccctgaagtgcctgagatacagattcaagaagcactgcaccctgtacaccgccgtgtccagcacctggttcgccttcagagatctgtgcatcgtgtaccgggacggcaacccctacgccgtgtgcgacaagtgcctgaagttctacagcaagatcagcggcatcctgggcttcgtgttcaccctgggcggcagcctgggcggcggcggcagcggcatcgtgggcatcgtggccggcctggccgtgctggccgtggtggtgatcggcgccgtggtggccaccgtgatgtgccggcggaaaagcagcggcggcaagggcggcagctacagccaggccgccagcagcgacagcgcccagggcagcgacgtgagcctgaccgcctgaagctcgctttcttgctgtccaatttctattaaaggttcctttgttccctaagtccaactactaaactgggggatattatgaagggccttgagcatctggattctgcctaataaaaaacatttattttcatgcagcaagctcgctttcttgctgtccaatttctattaaaggttcctttgttccctaagtccaactactaaactgggggatattatgaagggccttgagcatctggattctgcctaataaaaaacatttattttcatgcagcaaataaa

SEQ ID NO: 72 - RNA, 2282 nt, synthetic construct
  misc_feature 1..2282: note = Synthetic
  source 1..2282: mol_type = other RNA
gggacatttgcttctgacacaactgtgttcactagcaacctcaaacagacaccatggggatcctgggcttcgtgtttaccctcgctgcctacatcctgaccgccttcaactccagccacaaggctgcctacaacacaacccccatcgtgcacctgaaggctgcctacaccctcaagtgcctgagatacaggttcaaagctgcctacaggctggagtgcgccatctactataaggctgcctacgtggttgagggccaggtcgactactatgctgcctacaagagcgccatcgtgacactgacctacgctgcctacctgaccgcccccacaggctgcatcaagaaagctgcctacacatacaccagcctcattatcctggtgttggctgcctacaccattcatgatattattctggaatgcgtggctgcctacctgctcattaggtgtatcaactgccagaaggctgcctacatcattctggagtgtgtgtactgcaaggctgcctactgcgtgtactgtaagcagcaactcctgagggctgcctacacaaccctggagcagcaatacaacaaggctgcctacgccgtgtgcgacaaatgtctgaagttctacgctgcctacgccgagcccgacagggctcactacaacatcgtgaccttctgttgcaaggctgcctacggcaccctgggaatcgtgtgtcccatttgcagccagaaggctgcctacttcgtgtacatccccctgtttttgattcacacccatgccaggttcctcataggacccggccctgggatcaggccccttctgctgtccgtgagcacctacacatctctcataattggacccggccctggggatagcacactcaggctgtgcgtgcagtccacccacgtcgacatcagaactcttttggaggacttactgatgggacccggccctgggtattgctacgaacagctgaacgactccagcgaggaggaggataccgacgaaatcgattaagagttgtgccaatgcacgttgcatcgagatggcgctccgtcttaagctccaaggcacctgaatgtgtagtgattcgtctagaaatagacgaatcatgctgatctcaggtgctcacttgattaagacggctgtttatctcgatgcgcagccttttggcacaaggtaggcaggatttcttgggacacgtcctcctcgctggagtcgttcagcctgacaatgaaagaggattctccgggtaagcatgtaaagtcctcctaacgcttgctgatggcaggtgtgatggactgcgtaggtcatctgagttagccttgtccagggccgggtccaattatgagaagatcagcactgctcacatcactatgaaaggcgataggcccaggcgtgaggtagatgaggagatactctggggtgtgcgcacttgaaagggggcgatacaccaagtaggcagccttctggctgcaaggcggtatcacgattccatacatgctctaggcacaacgcatggaaattcacgcgttgttgtcgcactataggtcgggctctcgtacacgagcgtcgcggtcagcacatttgtcgcacacggcgtaggctgctatccggtattgcatcaagtgacttgtgtttacctcacacaggagaatgtcctagaaatcccctaaagaggcacaagatttgaacacacgttgaacctagatgtaggcagccttctggcagtttgtacacggaatgagcactcttaatgccacgctaagcgttacaatatcattatagtgccaggcagctgacggaagaaacggtaccgtggtgtatcttgatcaggctttcttgatgcagcctgtgggggaacttcagtaggcaggacgcacagctgtcaccataaacaggttgtagggagagtatcggtcgacgcaaatcttgaccacgtcgcgcgttgaaagcctttcctggcactccagcctgtaggcagcactccagaaatatctcagatcaaaaagggtgtagaacgtgcataggtgcaacacacctgttgtgttttgtacgagcttgtggcgtgatcaagtggcggtcaggattctagagcggccgcttcgagccggttgaatcgctgatctcacgccgtggtgagctcgctttcttgctgtccaatttctattaaaggttcctttgttccctaagtccaactactaaactgggggatattatgaagggccttgagcatctggattctgcctaataaaaaacatttattttcattgcaaagttccgcgtacgtacggcgtc

SEQ ID NO: 73 - RNA, 550 nt, synthetic construct
  misc_feature 1..550: note = Synthetic
  source 1..550: mol_type = other RNA
acatttgcttctgacacaactgtgttcactagcaacctcaaacagacaccacatttgcttctgacacaactgtgttcactagcaacctcaaacagacaccgccgccaccatgggcatcctgggcttcgtgttcaccctgtagagctcgctttcttgctgtccaatttctattaaaggttcctttgttccctaagtccaactactaaactgggggatattatgaagggccttgagcatctggattctgcctaataaaaaacatttattttcatgcagcaagctcgctttcttgctgtccaatttctattaaaggttcctttgttccctaagtccaactactaaactgggggatattatgaagggccttgagcatctggattctgcctaataaaaaacatttattttcatgcagcaaataaaagctcgctttcttgctgtccaatttctattaaaggttcctttgttccctaagtccaactactaaactgggggatattatgaagggccttgagcatctggattctgcctaataaaaaacatttattttcat

SEQ ID NO: 74 - RNA, 1452 nt, synthetic construct
  misc_feature 1..1452: note = Synthetic
  source 1..1452: mol_type = other RNA
gatcgacaagacttaaccacgattcctgatgcattgacttaccatcgactcaactgacaagggaccacgcagaggtgaatgagtcaggactttgtagtcggagtcggaaaaaacaccagtcacaatgtatcgtacgcttgctactaggagctcgtcatgacgttgagagcctgttaactagacacgttcctaagggttagccacacattaatatcgggcctgacacaggacacgaaaaaagaaggtgctgttagttggacaggtactatcatctcaagtcgatagtccaagtaggtttgaaccatgcatagcttgtatcaggtcatcgcctcaaacgttaggtgtcacattgtggaatcgcaaaaaacataccgacttccattatgggacacgtcgcttattcttggtaagtagaagttgccatcgtagtcgcacgacctacttatgacgaacttcggttaagtggctgacgtactaacagtgcgtgcaaaaaagacctacgaagccagagttcgttccagtgtgaaagtgcacatcacgagttgtgccaatgcacgttgcatcgagagttaatcccgtcttaagtagcaaggcacctgaatggaagttgattcgtctagaaatagacgaatcatgctgatctcaggtgctcacttgattaagacggctgtttatctcgatgcttcccccttggcacaaatgcatcagtgcacttatgatagtgaacgaactctggcttcgtaggtcaaaaaagcacgcaagcatgtaacgtcagcctaacgcttgaagttcgcaggtgtgaggtcgtgctttgtgtggcaactgtcatgacccaagaataagcgacgtgtcccatagatcagcacggtatgaaaaaagcgattcgtgaggtagacacctagatactctggcgatgactcggccagctatgcgagtcgataacctacttggactatcgacttgagtcacactgacctgtccatacatgctcaccttcaaaaaacgtgtccgcactatagcccgatatctcgtacaggctaaccttcttcgtgtctagttaacaggctctcaactctacttagagctcctatcaagtgacgtacgattacctcacactggtgaaaaaaccgactcaagatttgaagtcctgggcgacctctgcgtggtcccttgtcagttatggttcaggtaagtcactcgtgatggaatcgtaagcgttacttgtcgattatagtgcgctgctaaaaaatgttcttgatcaggcgtccgtgctcggccccttaggaatcccccggactacgattggctcataaacagggcctagagtatcttggccaaatcttgcggcaaaaaactgttgttttttttactcattctccgtgcctgatacacttctaacgtgcagtttgtcacacctgtgggctgtacgagcttttgtgatcaagcgcggcg

SEQ ID NO: 75 - RNA, 562 nt, synthetic construct
  misc_feature 1..562: note = Synthetic
  source 1..562: mol_type = other RNA
acatttgcttctgacacaactgtgttcactagcaacctcaaacagacaccacatttgcttctgacacaactgtgttcactagcaacctcaaacagacaccgccgccaccatgcccaagtacgtgaagcagaacaccctgaagctggccacctagagctcgctttcttgctgtccaatttctattaaaggttcctttgttccctaagtccaactactaaactgggggatattatgaagggccttgagcatctggattctgcctaataaaaaacatttattttcatgcagcaagctcgctttcttgctgtccaatttctattaaaggttcctttgttccctaagtccaactactaaactgggggatattatgaagggccttgagcatctggattctgcctaataaaaaacatttattttcatgcagcaaataaaagctcgctttcttgctgtccaatttctattaaaggttcctttgttccctaagtccaactactaaactgggggatattatgaagggccttgagcatctggattctgcctaataaaaaacatttattttcat

SEQ ID NO: 76 - RNA, 1443 nt, synthetic construct
  misc_feature 1..1443: note = Synthetic
  source 1..1443: mol_type = other RNA
cttaaccacgattcctgatgcattgacttaccatcgactcaactgacaagggaccacgcagaggtgaatgagtcaggactttgtagtcggagtcggaaaaaacaccagtcacaatgtatcgtacgcttgctactaggagctcgtcatgacgttgagagcctgttaactagacacgttcctaagggttagccacacattaatatcgggcctgacacaggacacgaaaaaagaaggtgctgttagttggacaggtactatcatctcaagtcgatagtccaagtaggtttgaaccatgcatagcttgtatcaggtcatcgcctcaaacgttaggtgtcacattgtggaatcgcaaaaaacataccgacttccattatgggacacgtcgcttattcttggtaagtagaagttgccatcgtagtcgcacgacctacttatgacgaacttcggttaagtggctgacgtactaacagtgcgtgcaaaaaagacctacgaagccagagttcgttccagtgtgaaagtgcacatcacgagttgtgccaatgcacgttgcatcgagagttaatcccgtcttaagtagcaaggcacctgaatggaagttgattcgtctagaaatagacgaatcatgctgatctcaggtgctcacttgattaagacggctgtttatctcgatgcttgtgtttggcacaaatgcatcagtgcacttatgatagtgaacgaactctggcttcgtaggtcaaaaaagcacgcaagcatgtaacgtcagcctaacgcttgaagttcgcaggtgtgaggtcgtgcgcttggtggcaactgtcatgacccaagaataagcgacgtgtcccatagatcagcacggtatgaaaaaagcgattcgtgaggtagacacctagatactctggcgatgacctttagctatgcgagtcgataacctacttggactatcgacttgagtcacactgacctgtccatacatgctcaccttcaaaaaacgtgtccgcactatagcccgatatctcgtacaggctaaccccccgttcgtgtctagttaacaggctctcaactctacttagagctcctatcaagtgacgtacgattacctcacactggtgaaaaaaccgactcaagatttgaagtcctggtgctcacctctgcgtggtcccttgtcagttatggttcaggtaagtcactcgtgatggaatcgtaagcgttatttttgcttatagtgcttgaaaaaattggccttgatcagtgctcggcccttctttccttaggaatgtgttggactacgagccataaacagtgtagagtatcggccaaatctttgctgaaaaaactgttttttttggcgtccactcattcggcccttctgatacacccccgtaacgtgcatggcttgcacacctgggcctttgtacgagttggcgtgatcaagcggctctggtg

SEQ ID NO: 77 - protein, 13 aa, synthetic construct
  REGION 1..13: note = Synthetic
GLFDIIKKIAESF

SEQ ID NO: 78 - protein, 10 aa, synthetic construct
  REGION 1..10: note = Synthetic
KKKKKKKKKK

SEQ ID NO: 79 - protein, 36 aa, synthetic construct
  REGION 1..36: note = Synthetic
GLFDIIKKIAESFKKKKKKKKKKGLFDIIKKIAESF

SEQ ID NO: 80 - protein, 10 aa, synthetic construct
  REGION 1..10: note = Synthetic
  SITE 10: note = MISC_FEATURE - C-terminal-PEG 5000 moiety
KKKKKKKKKK

SEQ ID NO: 81 - protein, 36 aa, synthetic construct; identical to SEQ ID NO: 79
  REGION 1..36: note = Synthetic
  SITE 13..14: note = MISC_FEATURE - PEG 1000 linker between F13 andK14
  SITE 23..24: note = MISC_FEATURE -PEG 1000 linker between K23 and G24